SEQUENCE LISTING (63 sequences)

SEQ ID NO: 1 - protein, 10 aa, Artificial Sequence
  synthetic construct
SFNSYELGSL

SEQ ID NO: 2 - protein, 10 aa, Artificial Sequence
  synthetic construct
TFNSYELGSL

SEQ ID NO: 3 - protein, 11 aa, Artificial Sequence
  synthetic construct
AFNSNYELGSL

SEQ ID NO: 4 - protein, 10 aa, Artificial Sequence
  synthetic construct
SFNSYELGTL

SEQ ID NO: 5 - protein, 10 aa, Artificial Sequence
  synthetic construct
STNSYELGSL

SEQ ID NO: 6 - protein, 10 aa, Artificial Sequence
  synthetic construct
SFNSFELGSL

SEQ ID NO: 7 - protein, 9 aa, Artificial Sequence
  synthetic construct
SNSYDLGSL

SEQ ID NO: 8 - protein, 10 aa, Artificial Sequence
  synthetic construct
SFNSYELPSL

SEQ ID NO: 9 - protein, 10 aa, Artificial Sequence
  synthetic construct
TFNSYELGTL

SEQ ID NO: 10 - protein, 10 aa, Artificial Sequence
  synthetic construct
SFNSYEIGSV

SEQ ID NO: 11 - protein, 10 aa, Artificial Sequence
  synthetic construct
SFNSYEVGSI

SEQ ID NO: 12 - protein, 10 aa, Artificial Sequence
  synthetic construct
SFNSYELGSV

SEQ ID NO: 13 - protein, 10 aa, Artificial Sequence
  synthetic construct
SFNSYELGSI

SEQ ID NO: 14 - protein, 10 aa, Artificial Sequence
  synthetic construct
SFNSYEIGSL

SEQ ID NO: 15 - protein, 10 aa, Artificial Sequence
  synthetic construct
SFNSYEVGSL

SEQ ID NO: 16 - protein, 10 aa, Artificial Sequence
  synthetic construct
AFNSYELGSL

SEQ ID NO: 17 - protein, 6 aa, Artificial Sequence
  synthetic construct
YDLGSL

SEQ ID NO: 18 - protein, 6 aa, Artificial Sequence
  synthetic construct
FDLGSL

SEQ ID NO: 19 - protein, 6 aa, Artificial Sequence
  synthetic construct
YDIGSL

SEQ ID NO: 20 - protein, 6 aa, Artificial Sequence
  synthetic construct
YDVGSL

SEQ ID NO: 21 - protein, 6 aa, Artificial Sequence
  synthetic construct
YDLPSL

SEQ ID NO: 22 - protein, 6 aa, Artificial Sequence
  synthetic construct
YDLGLL

SEQ ID NO: 23 - protein, 6 aa, Artificial Sequence
  synthetic construct
YDLGSI

SEQ ID NO: 24 - protein, 6 aa, Artificial Sequence
  synthetic construct
YDLGSV

SEQ ID NO: 25 - protein, 4 aa, Artificial Sequence
  synthetic construct
IGSL

SEQ ID NO: 26 - protein, 4 aa, Artificial Sequence
  synthetic construct
VGSL

SEQ ID NO: 27 - protein, 4 aa, Artificial Sequence
  synthetic construct
LPSL

SEQ ID NO: 28 - protein, 4 aa, Artificial Sequence
  synthetic construct
LGLL

SEQ ID NO: 29 - protein, 4 aa, Artificial Sequence
  synthetic construct
LGSI

SEQ ID NO: 30 - protein, 4 aa, Artificial Sequence
  synthetic construct
LGSV

SEQ ID NO: 31 - protein, 11 aa, Artificial Sequence
  synthetic construct
ALSTERGKTLV

SEQ ID NO: 32 - protein, 11 aa, Artificial Sequence
  synthetic construct
ALSTDRGKTLV

SEQ ID NO: 33 - protein, 11 aa, Artificial Sequence
  synthetic construct
ALTSDRGKTLV

SEQ ID NO: 34 - protein, 11 aa, Artificial Sequence
  synthetic construct
ALTTDRGKSLV

SEQ ID NO: 35 - protein, 11 aa, Artificial Sequence
  synthetic construct
ALTTDRPKTLV

SEQ ID NO: 36 - protein, 11 aa, Artificial Sequence
  synthetic construct
ALTTDRGRTLV

SEQ ID NO: 37 - protein, 11 aa, Artificial Sequence
  synthetic construct
ALTTDKGKTLV

SEQ ID NO: 38 - protein, 11 aa, Artificial Sequence
  synthetic construct
ALTTDKGKTLV

SEQ ID NO: 39 - protein, 10 aa, Artificial Sequence
  synthetic construct
VLMRAAEEPV

SEQ ID NO: 40 - protein, 10 aa, Artificial Sequence
  synthetic construct
QSMRSEDEAK

SEQ ID NO: 41 - protein, 9 aa, Artificial Sequence
  synthetic construct
AFNSYELGS

SEQ ID NO: 42 - protein, 9 aa, Artificial Sequence
  synthetic construct
QGFEKKTGV

SEQ ID NO: 43 - protein, 9 aa, Artificial Sequence
  synthetic construct
DNNGTYGKI

SEQ ID NO: 44 - protein, 6 aa, Artificial Sequence
  synthetic construct
KNLIDS

SEQ ID NO: 45 - protein, 8 aa, Artificial Sequence
  synthetic construct
VKSPRDYS

SEQ ID NO: 46 - protein, 9 aa, Artificial Sequence
  synthetic construct
VKSPCRDYS

SEQ ID NO: 47 - protein, 8 aa, Artificial Sequence
  synthetic construct
IKSPRLYS

SEQ ID NO: 48 - protein, 6 aa, Artificial Sequence
  synthetic construct
KNLIDS

SEQ ID NO: 49 - protein, 11 aa, Artificial Sequence
  synthetic construct
PKVKSPRDYSN

SEQ ID NO: 50 - protein, 11 aa, Artificial Sequence
  synthetic construct
YGRKKRRQRRR

SEQ ID NO: 51 - protein, 12 aa, Artificial Sequence
  synthetic construct
CYGRKKRRQRRR

SEQ ID NO: 52 - protein, 11 aa, Artificial Sequence
  synthetic construct
CSFNSYELGSL

SEQ ID NO: 53 - protein, 12 aa, Artificial Sequence
  synthetic construct
CYGRKKRRQRRR

SEQ ID NO: 54 - protein, 7 aa, Artificial Sequence
  synthetic construct
CKLFIMN

SEQ ID NO: 55 - protein, 20 aa, Artificial Sequence
  synthetic construct
XKLFIMNLGYGRKKRRQRRX

SEQ ID NO: 56 - protein, 21 aa, Artificial Sequence
  synthetic construct
XKLFIMNLSGYGRKKRRQRRX

SEQ ID NO: 57 - protein, 21 aa, Artificial Sequence
  synthetic construct
XEEVIRNISGYGRKKRRQRRX

SEQ ID NO: 58 - protein, 21 aa, Artificial Sequence
  synthetic construct
EAVSLKPTGGYGRKKRRQRRR

SEQ ID NO: 59 - protein, 21 aa, Artificial Sequence
  synthetic construct
XAVSLKPTGGYGRKKRRQRRR

SEQ ID NO: 60 - protein, 21 aa, Artificial Sequence
  synthetic construct
EAVSLKPTGGYGRKKRRQRRX

SEQ ID NO: 61 - protein, 21 aa, Artificial Sequence
  synthetic construct
XAVSLKPTGGYGRKKRRQRRX

SEQ ID NO: 62 - protein, 10 aa, Artificial Sequence
  synthetic construct
CSFNSYELGS

SEQ ID NO: 63 - protein, 11 aa, Artificial Sequence
  synthetic construct
CELGSLQAEDD

(19) United States
(12) Patent Application Publication
(10) Pub. No.: US 2009/0318351 A1
(43) Pub. Date: Dec. 24, 2009

(54) METHOD OF TREATING ACUTE ST-ELEVATION MYOCARDIAL INFARCTION WITH A DELTA PKC ANTAGONIST

(76) Inventors: Kevin Grimes, Mountain View, CA (US); Leon E. Chen, Belmont, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/403,302

(22) Filed: Mar. 12, 2009

Related U.S. Application Data
(60) Provisional application No. 61/035,912, filed on Mar. 12, 2008.

(51) Int. Cl. A61K 38/16

(52) U.S. Cl. 514/12

(57) ABSTRACT

Compositions, kits and methods are provided for preventing or treating tissue injury caused by stresses or shocks related to oxygen deficiency, such as ischemia, anoxia, hypoxia, reperfusion and other environmental stresses. In particular, protein kinase C (PKC) isozyme-specific agonists and antagonists are provided for treating cardiac tissue damage due to ischemia-reperfusion, such as acute myocardial infarction.

### RELATED APPLICATIONS

This application claims the benefit of priority of U.S. Provisional Application No. 61/035,912, filed Mar. 12, 2008 which is incorporated by reference in its entirety.

### FIELD OF THE INVENTION

The present invention relates to the use of delta PKC antagonist peptides, therapeutic peptide compositions and methods for the treatment of ST-elevation myocardial infarction.

### BACKGROUND

Protein kinase C (PKC) is a family of 11 different intracellular enzymes, also termed isozymes. Every cell type in the body expresses several different PKC isozymes. Individual PKC isozymes move, or translocate, to a unique subcellular location upon activation and that localization determines functional specificity (Mochly-Rosen et al. (1990) Cell Regul 1:693-706). Anchoring proteins termed RACKs (Receptors for Activated C Kinase) mediate PKC isozyme localization and each PKC isozyme binds selectively to one RACK (Mochly-Rosen et al. (1995) Science 268:247-251; Mochly-Rosen et al. (1991) J Biol Chem 266:1466-1486; Mochly-Rosen et al. (1991 Proc Natl Acad Sci USA 88:3997-4000).

Examples of PKC isozyme-specific agonists and antagonists are described in detail in U.S. Pat. Nos. 5,519,003; 6,262,023; 6,342,368; 6,423,684; 6,165,977; 5,776,716; 5,783,405; 5,935,803; and 6,054,286; in U.S. patent application Ser. Nos. 10/007,363; 10/007,761; 10/421,548; 10/428,280; and 12/017,985; and in PCT Application No. PCT/US2008/074509. These patents and applications are incorporated herein by reference in their entirety.

It has been reported that KAI-9803 (also known as δV1-1) inhibits δPKC activity by disrupting binding of δPKC to its receptor for activated C kinase, thereby preventing localization of δPKC to the mitochondria during periods of myocardial ischemia and reperfusion. δPKC inhibition during the reperfusion period leads to restoration of cellular energy stores, enhanced recovery of intracellular acidosis, preservation of mitochondrial function, and, ultimately, reduced damage to myocytes and endothelial cells after an ischemic insult. In pre-clinical studies, when given as a single intracoronary dose just prior to reperfusion, KAI-9803 was found to reduce infarct size, enhance early recovery of regional left ventricular contractility, and improve microvascular patency and function in animal models of acute myocardial infarction.

The present invention provides PKC agonist or antagonist peptides, therapeutic peptide compositions and methods for treating diseases or conditions. In particular, the present invention provides a method is provided for treating acute STEMI. In a typical embodiment, the method comprises: administering to a patient suffering from STEMI a therapeutically effective amount of a PKC isozyme-specific antagonist, such as a δPKC antagonist.

### SUMMARY OF THE INVENTION

Acute ST-segment elevation myocardial infarction (STEMI) is commonly caused by atherosclerotic plaque rupture which results in thrombotic occlusion of the epicardial coronary vessel and subsequent myocardial cellular ischemia and necrosis. While rapid epicardial reperfusion with percutaneous coronary intervention (PCI) has been shown to lower mortality and improve patient outcomes, successful epicardial reperfusion is often limited by microvascular dysfunction of the infarct territory that disrupts myocardial tissue reperfusion and limits the efficacy of reperfusion therapies in at least 25-50% of patients (Roe et al. (2001) J Am Coll Cardiol 37:9-18). Microvascular dysfunction is thought to be caused by reperfusion injury following restoration of blood flow to the infarct zone which causes tissue inflammation, free radical generation, and endothelial dysfunction, as well as microvascular obstruction from embolization of thrombotic material (Kloner et al. (1993) J Am Coll Cardiol 21:537-45).

The delta PKC inhibitory peptide KAI-9803 was evaluated as an adjunctive therapy for patients with STEMI in the Direct Inhibition of δ Protein Kinase C Enzyme to Limit Total Infarct Size in Acute Myocardial Infarction (DELTA MI) trial. In the DELTA MI trial, the safety, tolerability, and activity of escalating doses of KAI-9803 were evaluated when administered by intracoronary injection during primary percutaneous coronary intervention (PCI) for acute anterior STEMI. Based on the results of the DELTA MI trial, it is contemplated that KAI-9803 and other delta PKC inhibitory peptides can be used for the treatment of STEMI.

KAI-9803 has been shown to ameliorate injury associated with ischemia and reperfusion in animal models of acute myocardial infarction (MI). DELTA MI trial was a dose escalation study that evaluated the safety, tolerability, and activity of KAI-9803 for patients with acute anterior STEMI undergoing PCI. Patients who presented within 6 hours of symptom onset and had an occluded left anterior descending infarct artery on angiography were randomized in a 2:1 fashion to receive 1 of 4 doses of KAI-9803 (cohort 1: 0.05 mg; cohort 2: 0.5 mg; cohort 3: 1.25 mg; cohort 4: 5.0 mg) vs. blinded concurrent placebo delivered in 2 divided doses via intracoronary injection before and after re-establishment of antegrade epicardial flow with PCI. Safety and biomarker end points were assessed. Overall, 154 patients were randomized and treated with study drug (37 in cohort 1, 38 in cohort 2, 38 in cohort 3, 41 in cohort 4). The incidence of serious adverse events was similar between patients treated with KAI-9803 vs. placebo. Other safety end points, including changes in QT intervals and standard laboratory values after study drug administration, were similar between treatment groups. The study was not powered to demonstrate efficacy with the biomarker end points assessed. However, evidence of drug activity with KAI-9803 were supported by trends for consistent, non-significant reductions in creatine kinase-MB area-under-the-curve (AUC) and ST recovery AUC values across all dosing cohorts with KAI-9803 compared with concurrent placebo while similar trends were demonstrated for improvements in Technetium Tc-99m Sestamibi infarct size values with active study drug in cohorts 1,2, and 3. Furthermore, KAI-9803 had an acceptable safety and tolerability profile when delivered via intracoronary injection during primary PCI for STEMI.

### BRIEF DESCRIPTION OF THE DRAWINGS

FIG. 1. Patient flow diagram detailing the composition of the modified intent-to-treat population and patients lost to follow-up (LFU) through 6 months.

FIGS. 2a and 2b. Continuous distribution curves representing data from the pooled active study drug and placebo populations. FIG. 2a shows CK-MB area-under-the-curve (AUC) data. FIG. 2b shows ST recovery AUC data.

FIGS. 3a, 3b, and 3c. Results from key biomarker end points for active study drug compared with concurrent placebo within each dosing cohort. FIG. 3a shows CK-MB area-under-the-curve (AUC) median values for each dose cohort. FIG. 3b shows ST recovery AUC median values for each dose cohort. FIG. 3c shows Technetium Tc-99m Sestamibi infarct size median values for each dose cohort.

FIG. 4A-4D shows graphs illustrating the impact of temperature on the stability KAI-9706 at 26° C. (4A) and 37° C. (4B), and on KAI 1455 at 26° C. (4C) and 37° C. (4D).

FIG. 5 shows a illustrates the cargo peptide of SEQ ID NO:52 with a cysteine residue at the amino terminus of the peptide.

FIG. 6A-B shows illustrate the conversion of a therapeutic peptide comprising a carrier peptide (CKLFIMN) (SEQ ID NO:54) and a cargo peptide (CYGRKKRRQRRR) (SEQ ID NO:53) linked via a disulfide bond (6A) to a therapeutic peptide (Ac-GKLFIMNLGYGRKKRRQRRR-NH₂) (SEQ ID NO:55) in linear form (6B). This linear peptide (KP-01547) has been capped at its amino and carboxy termini and contains a short amino acid sequence linker.

FIG. 7 shows illustrated two linear peptides (Ac-GKLFIMNLSGYGRKKRRQRRR-NH₂)((SEQ ID NO:56) and (Ac-GEEVIRNISGYGRKKRRQRRR-NH₂) (SEQ ID NO:57).

FIG. 8 shows a graph comparing the stability of various PKC-β_{I} therapeutic peptides over time.

FIG. 9 shows a graph a graph comparing the stability of various PKC-β_{II}, therapeutic peptides over time.

FIG. 10A-10D show four different possible configurations of therapeutic peptide multimers.

FIG. 11 shows a graph plotting CK release against concentrations of therapeutic peptides KAI-9706 and KAI-1455.

FIG. 12 shows a graph plotting percent infarction against increasing concentrations of therapeutic peptides KAI-9706 and KAI-1455.

FIGS. 13A-13B show graphs plotting percent of intact therapeutic peptides KAI-9803, KAI-9706, and KAI-1455, surviving over time in human serum (3A), pig serum (3B), and rat serum (3C).

FIG. 14 shows a graph plotting percent of intact therapeutic peptides (linear and non-linear) over time (days).

FIG. 15 shows a graph comparing the stability of therapeutic peptides over time (days).

FIG. 16 shows a graph plotting creatine kinase release in the presence of increasing concentrations of KAI-9803 or KAI-1355 peptides in a Langendorff in vitro post-ischemia assay, with 10 minutes perfusion.

FIG. 17 shows an illustration depicting the construction of KAI-1479.

FIGS. 18A and 18B show bar graphs of results from a reperfusion study, wherein 18A illustrates the time line of events during the experiment and 18B shows a bar graph illustrating the protective properties of various therapeutic peptides (KAI-9803, KAI-1479, and KAI-1482).

FIG. 19 shows linear peptides KP-1680(EAVSLKPTGGYGRKKRRQRRR) (SEQ ID NO:58), KP-1681(Ac-EAVSLKPTGGYGRKKRRQRRR) (SEQ ID NO:59), KP-1633(EAVSLKPTGGYGRKKRRQRRR-NH₂) (SEQ ID NO:60), and KP-1678(Ac-EAVSLKPTGGYGRKKRRQRRR-NH₂) (SEQ ID NO:61).

FIG. 20A-D shows graphs of the percent of peptides remaining in test solutions over time.

### DETAILED DESCRIPTION OF THE INVENTION

The present invention provides innovative compositions, kits and methods for preventing or treating tissue injury caused by stresses or shocks related to oxygen deficiency. Such stresses include ischemia, anoxia, hypoxia, reperfusion and other environmental stresses related to a lack of sufficient oxygen, causing damages to tissues such as cardiac, skeletal muscle or nerve tissues.

Specific clinical indications of these types of stress include, but are not limited to, ischemia-reperfusion, transplantation, stroke, reconstructive surgery, septic shock, acute respiratory distress syndrome (ARDS), frost bite, hypoperfusion, acute lung injury, etc. In particular, the present invention provides compositions and methods for preventing or treating cardiac tissue injury due to ST-elevation myocardial infarction (STEMI).

As used herein, the term “Anoxia” refers to a virtually complete absence of oxygen in the organ or tissue, which, if prolonged, may result in death of the cell, organ or tissue. The term “Hypoxia” refers to a condition under which a cell, organ or tissue receive an inadequate supply of oxygen. “Reperfusion” refers to return of fluid flow into a tissue after a period of no-flow or reduced flow. For example, in reperfusion of the heart, fluid or blood returns to the heart through the coronary arteries after occlusion of these arteries has been removed. The term “Tissue” as used herein intends a whole organ, either in vivo or ex vivo, a fragment of an organ, or two or more cells.

In one embodiment of the invention, a composition is provided that is adapted for administration to a human. The composition comprises a protein kinase C (PKC) isozyme-specific antagonist (that inhibits the PKC).

In another embodiment, the invention relates to methods of modifying peptide compositions to increase stability and delivery efficiency. Specifically, the invention relates to methods to increase the stability and delivery efficiency of protein kinase C (PKC) modulatory peptide compositions. A “therapeutic peptide composition” comprises a “carrier peptide” and a “cargo peptide.” A “carrier peptide” is a peptide or amino acid sequence within a peptide that facilitates the cellular uptake of the therapeutic peptide composition. The “cargo peptide” is a PKC modulatory peptide. Peptide modifications to either the carrier peptide, the cargo peptide, or both, which are described herein increase the stability and delivery efficiency of therapeutic peptide compositions by reducing disulfide bond exchange, physical stability, reducing proteolytic degradation, and increasing efficiency of cellular uptake.

### Disulfide Bond Exchange,

A preferred embodiment of the disclosed therapeutic peptide compositions provides a cargo peptide coupled to a carrier peptide via a disulfide bond between two joining sulfur-containing residues, one in each peptide. The disulfide bond of this embodiment can be unstable whether the therapeutic peptide composition is in solution, lyophilized, precipitated, crystallized, or spray-dried, leading to carrier-cargo combinations to degrade to carrier-carrier compositions, which are inactive, and cargo-cargo compositions, which are also inactive and are frequently insoluble. The stability of the disclosed therapeutic peptide compositions is improved through the use of chemical modifications and by controlling the physical environment of the peptide compositions prior to use.

### Chemical Modifications

The joining sulfur-containing residue can be placed anywhere in the sequence of the carrier or cargo peptides. For example, a preferred embodiment of the disclosed therapeutic peptide composition typically has the joining sulfur-containing residue at the amino terminus of the carrier and cargo peptides. The joining sulfur-containing residues can be placed at the carboxy termini of the peptides, or alternatively at the amino terminus of peptide and at the carboxy terminus of the other peptide. Additionally, the joining sulfur-containing residue can be placed anywhere within the sequence of either or both of the peptides. Placing the joining sulfur-containing residue within the carrier peptide, the cargo peptide, or both has been observed to reduce the rate of disulfide bond exchange.

An example of chemical modifications useful to stabilize the disulfide bonds of the therapeutic peptide compositions involves optimizing the amino acid residue or residues immediately proximate to the sulfur-containing residues used to join the carrier and cargo peptide. A preferred method of stabilizing the disulfide bond involves placing an aliphatic residue immediately proximate to the sulfur-containing residue in the carrier and/or cargo peptides. Aliphatic residues include alanine, valine, leucine and isoleucine. Thus, when the joining sulfur-containing residue is placed at the amino terminus of a peptide, an aliphatic residue is placed at the penultimate amino terminal position of the peptide to reduce the rate of disulfide bond exchange. When the joining sulfur-containing residue is located at the carboxy terminus of a peptide, an aliphatic residue is placed at the penultimate carboxy terminal position of the peptide to reduce the rate of disulfide bond exchange. When the joining sulfur-containing residue is located within the sequence of a peptide, the aliphatic residue can be place at either the amino terminal or carboxy terminal side of the residue, or at both sides.

A variety of sulfur-containing residues are contemplated for use with the presently disclosed invention. Cysteine and cysteine analogs can also be used as the joining cysteine residues in the peptide composition. Particular cysteine analogs include D-cysteine, homocysteine, alpha-methyl cysteine, mercaptopropionic acid, mercaptoacetic acid, penicillamine, acetylated forms of those analogs capable of accepting an acetyl group, and cysteine analogs modified with other blocking groups. For example, the use of homocysteine, acetylated homocysteine, penicillamine, and acetylated penicillamine in the cargo, the carrier, or both peptides have been shown to stabilize the peptide composition and decrease disulfide bond exchange. Alpha-methyl cysteine inhibits disulfide degradation because the base-mediated abstraction of the alpha hydrogen from one cysteine is prevented by the presence of the sulfur atom. Cargo/carrier peptide conjugates joined by disulfide bonds have been shown to be more resistant to glutathione reduction than unmodified peptides. Other cysteine analogs are also useful as joining cysteines. Similarly, stereoisomers of cysteine will inhibit disulfide bond exchange.

Disulfide bond exchange can be eliminated completely by linking the carrier and cargo peptides to form a single, linear peptide. This method is discussed below.

### Physical Stability

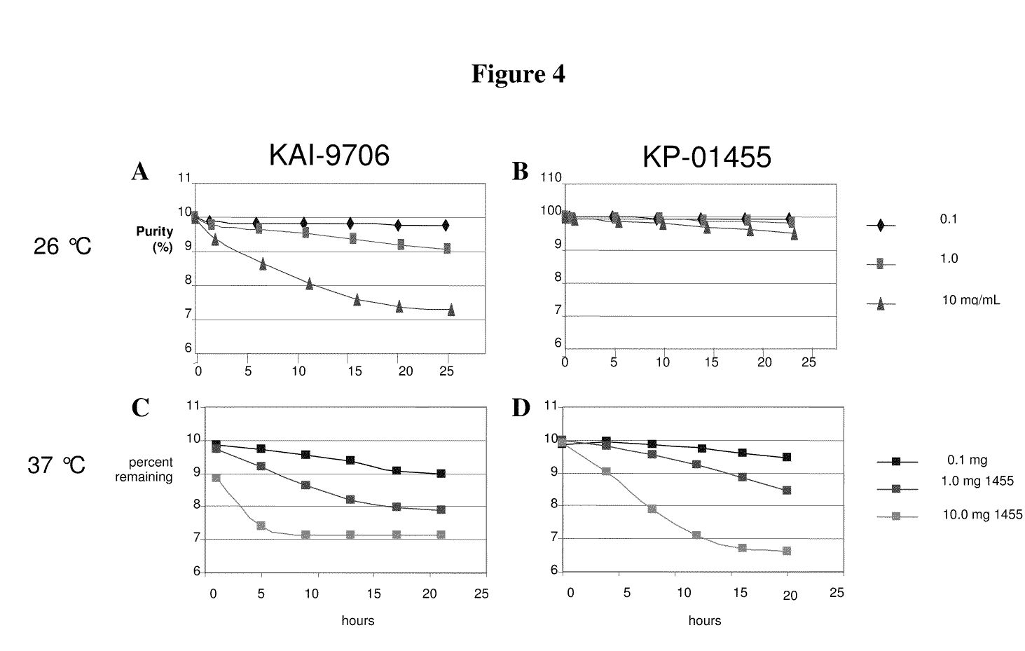

The physical environment of the disulfide has an effect on stability. As shown (in part) in FIG. 4, stability increases in solution as the pH of the solution decreases (acidic environment better than basic), the temperature of the solution decreases, and as the concentration of the peptide composition in solution decreases. In the lyophilized form, stability increases as the pH decreases, the temperature decreases, and the ratio of the peptide composition to excipient increases. Preferred excipients are discussed in U.S. patent application Ser. No. 11/240,962, filed Sep. 30, 2005, which is hereby incorporated by reference in its entirety.

The unexpected “excipient effect” was most pronounced for mannitol, which is a highly crystalline excipient. Using less crystalline excipients (such as sucrose) or even using no excipient, showed much less dependency on peptide composition quantity. Although not wishing to be bound or limited by any theory, it is thought that use of a non-crystalline excipient creates an amorphous matrix, which helps prevent intermolecular associations. Theoretically, in a crystalline matrix the peptide composition is excluded and forced to the walls of the vial, perhaps causing high local concentrations. With low amount of API the resulting thin film has high peptide-glass contact area and the silica is destabilizing.

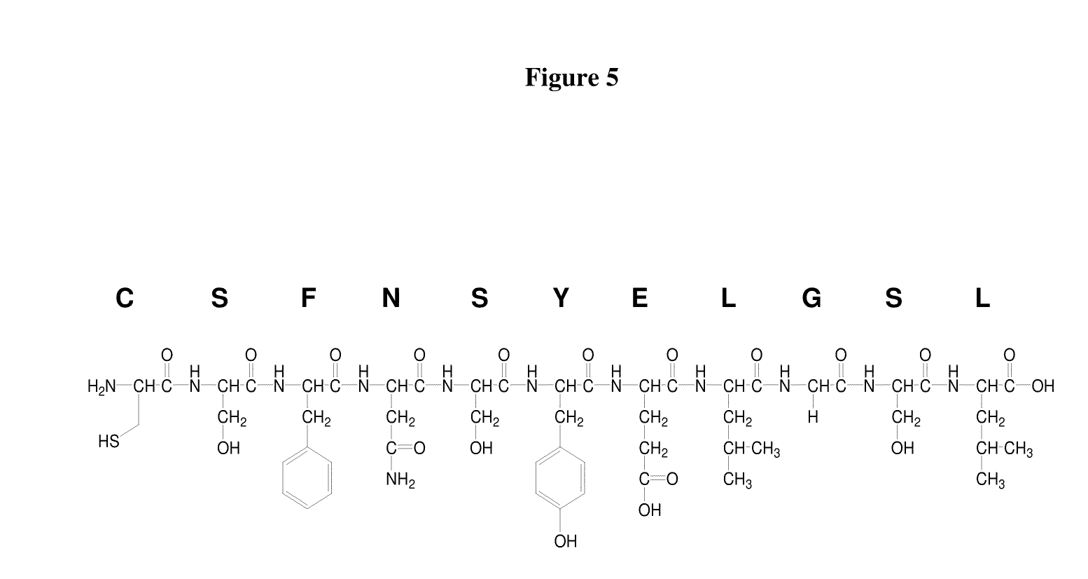

A number of factors impact the efficiency with which a therapeutic peptide composition is taken up by a target cell. For example, the solubility of a therapeutic peptide impacts the efficiency with which the peptide is taken up by a target cell. In turn, the amino acid sequence of a carrier or cargo peptide largely determines that solubility the peptide compositions in which they are used. Some peptides, particularly cargo peptides, will contain hydrophobic residues, (e.g., Phe, Tyr, Leu), with regular spacing which allows for intramolecular interactions by a “zipper” mechanism leading to aggregation. An example of such a potentially problematic peptide is shown in FIG. 5. The illustrated sequence is believed to form a beta-strand in the V1 domain of δPKC. Such peptides have the tendency to form insoluble deposits.

The solubility of such peptides can be improved by making certain modifications to the cargo peptide sequence. For example, the introduction of solubilizing groups at amino and or carboxy termini or on internal residues, such as hydrating groups, like polyethylene glycol (PEG), highly charged groups, like quaternary ammonium salts, or bulky, branched chains of particular amino acid residues will improve the solubility of peptides like the one illustrated in FIG. 5. Additionally, those hydrophobic side chains that are shown not to be required for activity can be eliminated by deletion or substitution with a conservative or non-interfering residue, such as an alanine, glycine, or serine, thus improving the solubility of the peptides.

### Proteolytic Degradation Plasma Stability

Blood and plasma contain proteases which can degrade the protein kinase C modulatory peptides disclosed herein or the carrier peptides which facilitate the cellular uptake of the peptide composition, or both. One method to decrease proteolytic degradation of the carrier or cargo peptides is to mask the targets of the proteases presented by the therapeutic peptide composition.

Once the therapeutic peptide enters the plasma of a subject, it become vulnerable to attack by peptidases. Strategies are provided which address peptide degradation caused by exopeptidases (any of a group of enzymes that hydrolyze peptide bonds formed by the terminal amino acids of peptide chains) or endopeptidases (any of a group of enzymes that hydrolyze peptide bonds within the long chains of protein molecules). Exopeptidases are enzymes that cleave amino acid residues from the amino or carboxy termini of a peptide or protein, and can cleave at specific or non-specific sites. Endopeptidases, which cleave within an amino acid sequence, can also be non-specific, however endopeptidases frequently recognize particular amino sequences (recognition sites) and cleaves the peptide at or near those sites.

One method of protecting peptide compositions from proteolytic degradation involves the “capping” the amino and/or carboxy termini of the peptides. The term “capping” refers to the introduction of a blocking group to the terminus of the peptide via a covalent modification. Suitable blocking groups serve to cap the termini of the peptides without decreasing the biological activity of the peptides. Acetylation of the amino termini of the described peptides is a preferred method of protecting the peptides from proteolytic degradation. Other capping moieties are possible. The selection of acylating moiety provides an opportunity to “cap” the peptide as well as adjust the hydrophobicity of the compound. For example, the hydrophobicity increases for the following acyl group series: formyl, acetyl, propanoyl, hexanoyl, myristoyl, and are also contemplated as capping moieties. Amidation of the carboxy termini of the described peptides is also a preferred method of protecting the peptides from proteolytic degradation.

Protecting peptides from endopeptidases typically involves identification and elimination of an endopeptidase recognition site from a peptide. Protease recognition cites are well known to those of ordinary skill in the art. Thus it is possible to identify a potential endoprotease recognition site and then eliminating that site by altering the amino acid sequence within the recognition site. Residues in the recognition sequence can be moved or removed to destroy the recognition site. Preferably, a conservative substitution is made with one or more of the amino acids which comprise an identified protease recognition site. The side chains of these amino acids possess a variety of chemical properties. For the purposes of the present discussion, the most common amino acids are categorized into 9 groups, listed below. Substitution within these groups is considered to be a conservative substitution.

Conservative Amino Acid Substitution

| | |
|---|---|
| Small/Aliphatic residues: | Gly, Ala, Val, Leu, Ile |
| Cyclic Imino Acid: | Pro |
| Hydroxyl Residues: | Ser, Thr |
| Acidic Residues: | Asp, Glu |
| Amide Residues: | Asn, Gln |
| Basic Residues: | Lys, Arg |
| Imidazole Residue: | His |
| Aromatic Residues: | Phe, Tyr, Trp |
| Sulfur-Containing Residues: | Met, Cys |

### Efficiency of Cellular Uptake

In addition to the modifications discussed above, improve utility for the disclosed therapeutic peptide compositions can be achieved by altering the linkage of the carrier and cargo peptides. For example, in one embodiment, carrier and cargo peptides are linked by a peptide bond to form a linear peptide. Stability and potency of the therapeutic peptides can also be increased through the construction of peptide multimers, wherein a plurality of cargo peptides is linked to one or more carrier peptides. An additional embodiment of the invention involving a cleavable linker sequence is also discussed.

### Linear Peptides

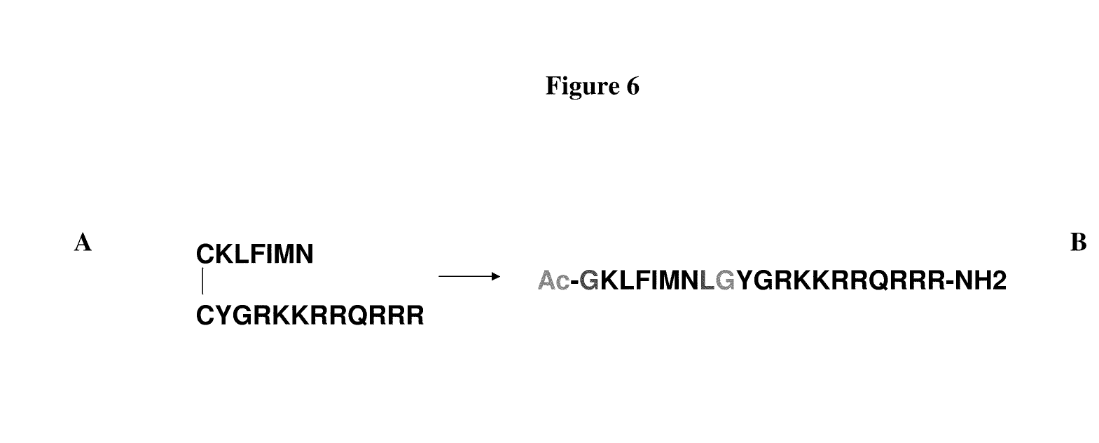

Another strategy to improve peptide composition stability involves joining the cargo and carrier peptides into a single peptide as opposed to joining the peptides via a disulfide bond. For example, in the embodiment shown in FIG. 6A, the cargo peptide (SEQ ID NO:13) linked via amino terminal cysteines. A linear version of the cargo and carrier peptides is shown in FIG. 6B, where the cargo and carrier peptides are linked via a short dipeptide linker (e.g., Ser-Gly). This linker is exemplary.

In the example illustrated, the C-terminus of cargo is linked to the N-terminus of the carrier via the linker. However, the other possible permutations are also contemplated, including linking the peptide via there C-termini, their N-termini, and where the carrier peptide is located at the N-terminal portion of the peptide composition.

Additionally, the steps discussed above to stabilize a disulfide bond linked peptide composition can also be used with a linear, where appropriate. For example, the linear peptide composition shown in FIG. 6B has been capped at both its amino and carboxy termini. Moreover sequences within the peptide can be scrambled or substituted with D-amino acids.

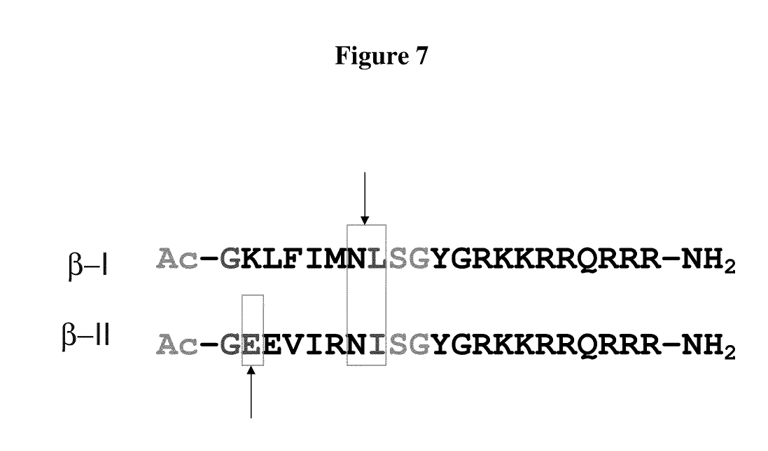

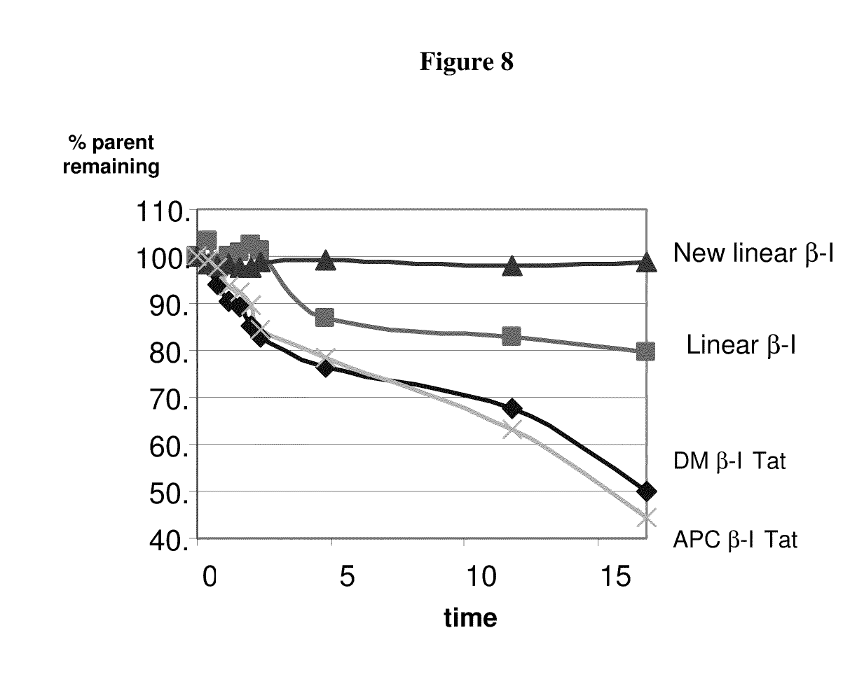

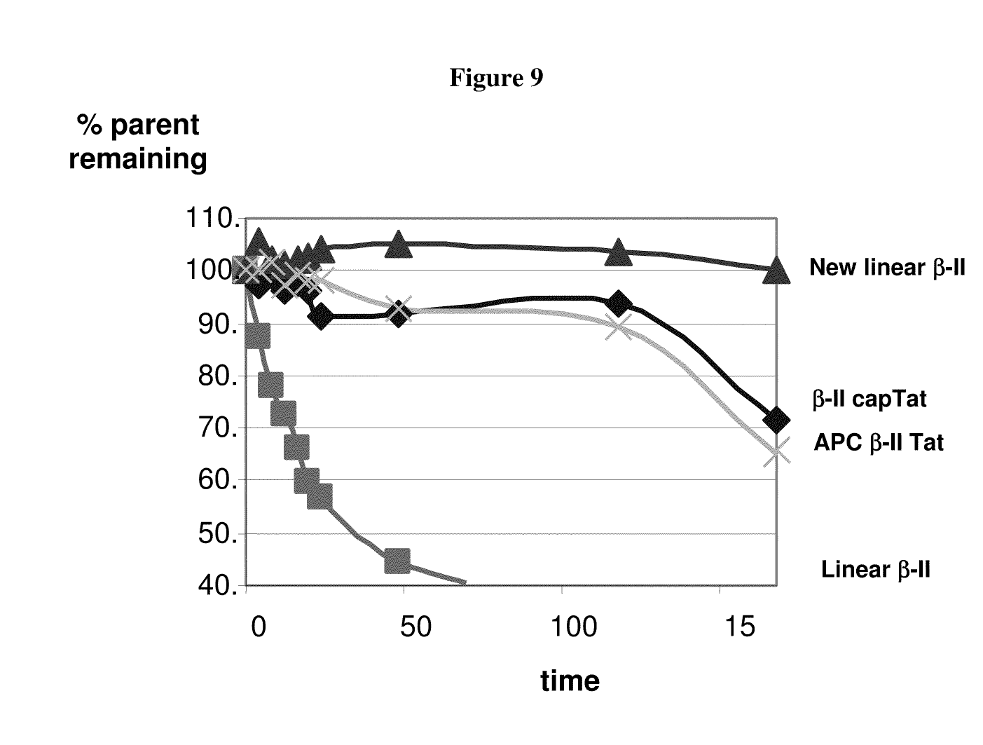

As shown in FIG. 7, deamination of Asn at position 7 of the β-I had been observed to cause significant instability in the linearized version of the peptide composition linked by Asn-Gly. Changing the Gly to Leu stabilized this linear peptide composition. Similarly, deamination of the Gln residue at position 2 of the linear β-II composition was observed to cause significant instability. Substitution with Glu improved stability of the linear composition. Data comparing the modified versions of these peptides is shown in FIGS. 8 and 9.

Without being limited to any particular theory, it is thought that deamination results from the attack of the alpha or main-chain amide HN-C-terminal to the Asn residue on the side-chain amide of Asn, generating the cyclic aspartamide intermediate which can hydrolyze to an aspartic acid moiety. Increasing the size of the residue C-terminal to Asn is thought to increase the steric hinderance on the main-chain amide, significantly slowing deamidation.

### Peptide Multimers

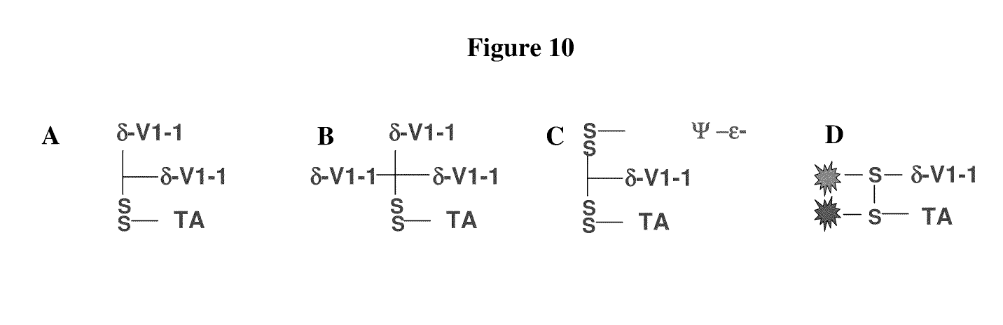

Another method of improving stability and potency is available by forming multimers with a plurality of cargo peptides associated with one or more carrier peptides. Examples of such formulations are shown in FIG. 10. Branched, multivalent peptide compositions will increase avidity, potency and stability of the compositions. By engineering cleavage sites or other release mechanisms into the multimer compositions, the multiple conjugates can release nearly simultaneously, PKC modulatory cargo peptides inside a target cell. An example of multimeric peptides is discussed in Yu et al. JBC 275(6):3943-9 (2000).

Cleavable Sequence

Typically the carrier and cargo are linked by a linkage that can be cleaved by ubiquitous enzymes such as esterases, amidases, and the like. It is assumed that the concentration of such enzymes is higher inside cells rather than in the extracellular milieu. Thus, once the conjugate is inside a cell, it is more likely to encounter an enzyme that can cleave the linkage between cargo and carrier. The enzyme can thus release the biologically active cargo inside a cell, where it presumably is most useful.

### Protein Kinase C Modulatory Peptides

The term protein kinase C modulatory peptide refers to a peptide derived from a PKC isozyme- and/or variable region. Various PKC isozyme- and variable region-specific peptides have been described and can be used with the presently disclosed invention. Preferably, the PKC modulatory peptide is a V1, V3 or V5-derived peptide. (The terminology “V1” and “C2” are synonymous.) The following US Patents or Patent Applications describe a variety of suitable peptides that can be used with the presently disclosed invention: U.S. Pat. Nos. 5,783,405, 6,165,977, 6,855,693, US2004/0204364, US2002/0150984, US2002/0168354, US2002/057413, US2003/0223981, US2004/0009922 and 10/428,280, each of which are incorporated herein by reference in their entirety. Table 1 provides a listing of preferred PKC modulatory peptides for use with the present invention.

TABLE 1
Cargo Peptides derived from PKC isozymes

| Peptide | SEQ ID NO. | Sequence |
|---|---|---|
| δV1-1.1 | SEQ ID NO: 1 | S-F-N-S-Y-E-L-G-S-L |
| δV1-1.2 | SEQ ID NO: 2 | T-F-N-S-Y-E-L-G-S-L |
| δV1-1.3 | SEQ ID NO: 3 | A-F-N-S-N-Y-E-L-G-S-L |
| δV1-1.4 | SEQ ID NO: 4 | S-F-N-S-Y-E-L-G-T-L |
| δV1-1.5 | SEQ ID NO: 5 | S-T-N-S-Y-E-L-G-S-L |
| δV1-1.6 | SEQ ID NO: 6 | S-F-N-S-F-E-L-G-S-L |
| δV1-1.7 | SEQ ID NO: 7 | S-N-S-Y-D-L-G-S-L |
| δV1-1.8 | SEQ ID NO: 8 | S-F-N-S-Y-E-L-P-S-L |
| δV1-1.9 | SEQ ID NO: 9 | T-F-N-S-Y-E-L-G-T-L |
| δV1-1.10 | SEQ ID NO: 10 | S-F-N-S-Y-E-I-G-S-V |
| δV1-1.11 | SEQ ID NO: 11 | S-F-N-S-Y-E-V-G-S-I |
| δV1-1.12 | SEQ ID NO: 12 | S-F-N-S-Y-E-L-G-S-V |
| δV1-1.13 | SEQ ID NO: 13 | S-F-N-S-Y-E-L-G-S-I |
| δV1-1.14 | SEQ ID NO: 14 | S-F-N-S-Y-E-I-G-S-L |
| δV1-1.15 | SEQ ID NO: 15 | S-F-N-S-Y-E-V-G-S-L |
| δV1-1.16 | SEQ ID NO: 16 | A-F-N-S-Y-E-L-G-S-L |
| δV1-1.17 | SEQ ID NO: 17 | Y-D-L-G-S-L |
| δV1-1.18 | SEQ ID NO: 18 | F-D-L-G-S-L |
| δV1-1.19 | SEQ ID NO: 19 | Y-D-I-G-S-L |
| δV1-1.20 | SEQ ID NO: 20 | Y-D-V-G-S-L |
| δV1-1.21 | SEQ ID NO: 21 | Y-D-L-P-S-L |
| δV1-1.22 | SEQ ID NO: 22 | Y-D-L-G-L-L |
| δV1-1.23 | SEQ ID NO: 23 | Y-D-L-G-S-I |
| δV1-1.24 | SEQ ID NO: 24 | Y-D-L-G-S-V |
| δV1-1.25 | SEQ ID NO: 25 | I-G-S-L |
| δV1-1.26 | SEQ ID NO: 26 | V-G-S-L |
| δV1-1.27 | SEQ ID NO: 27 | L-P-S-L |
| δV1-1.28 | SEQ ID NO: 28 | L-G-L-L |
| δV1-1.29 | SEQ ID NO: 29 | L-G-S-I |
| δV1-1.30 | SEQ ID NO: 30 | L-G-S-V |
| δV1-2 | SEQ ID NO: 31 | A-L-S-T-E-R-G-K-T-L-V |
| δV1-2.1 | SEQ ID NO: 32 | A-L-S-T-D-R-G-K-T-L-V |
| δV1-2.2 | SEQ ID NO: 33 | A-L-T-S-D-R-G-K-T-L-V |
| δV1-2.3 | SEQ ID NO: 34 | A-L-T-T-D-R-G-K-S-L-V |
| δV1-2.4 | SEQ ID NO: 35 | A-L-T-T-D-R-P-K-T-L-V |
| δV1-2.5 | SEQ ID NO: 36 | A-L-T-T-D-R-G-R-T-L-V |
| δV1-2.6 | SEQ ID NO: 37 | A-L-T-T-D-K-G-K-T-L-V |
| δV1-2.7 | SEQ ID NO: 38 | A-L-T-T-D-K-G-K-T-L-V |
| δV1-3 | SEQ ID NO: 39 | V-L-M-R-A-A-E-E-P-V |
| δV1-4 | SEQ ID NO: 40 | Q-S-M-R-S-E-D-E-A-K |
| δV1-5 | SEQ ID NO: 41 | A-F-N-S-Y-E-L-G-S |
| δV3-1 | SEQ ID NO: 42 | Q-G-F-E-K-K-T-G-V |
| δV3-2 | SEQ ID NO: 43 | D-N-N-G-T-Y-G-K-I |
| δV5-1 | SEQ ID NO: 44 | K-N-L-I-D-S |
| δV5-2 | SEQ ID NO: 45 | V-K-S-P-R-D-Y-S |
| δV5-2.1 | SEQ ID NO: 46 | V-K-S-P-C-R-D-Y-S |
| δV5-2.2 | SEQ ID NO: 47 | I-K-S-P-R-L-Y-S |
| δV5-3 | SEQ ID NO: 48 | K-N-L-I-D-S |
| δV5-4 | SEQ ID NO: 49 | P-K-V-K-S-P-R-D-Y-S-N |

A particular example of a PKC antagonist peptide is the δV1-1 peptide (amino acid sequence: SFNSYELGSL) (SEQ ID NO:1) which is a δPKC-specific antagonist.

PKC antagonist peptides can be conjugated to a carrier moiety that is effective to facilitate transport of the peptides across a cell membrane. Examples of the carrier moiety include, but are not limited, a Tat-derived peptide, an Antennapedia derived peptide, and a polyarginine peptide, such as octa-Arg or octa-D-Arg. Table 2 provides a listing of preferred carrier peptides for use with the present invention.

TABLE 2
Carrier Peptides

| | | |
|---|---|---|
| TAT Carrier | SEQ ID NO: 50 | YGRKKRRQRRR |
| Peptide | | |
| TAT Carrier | SEQ ID NO: 51 | CYGRKKRRQRRR |
| Peptide with N- | | |
| terminal Cys | | |

For example, the δV1-1 peptide can be linked to a Tat-derived peptide (amino acid sequence: YGRKKRRQRRR) (SEQ ID NO:50) via a cysteine-cysteine disulfide bond at their N termini (structure shown below), resulting in the molecule named KAI-9803 described herein.

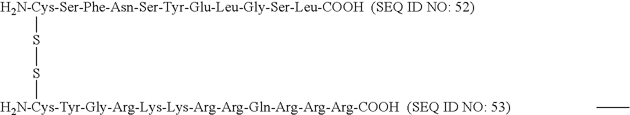

KAI-9803 inhibits δPKC activity by disrupting binding of δPKC with its RACK. KAI-9803 mimics the RACK binding-site on δPKC and, therefore, binds selectively to the δRACK at the corresponding δPKC binding-site, competitively inhibiting the binding of δPKC. By preventing δPKC from attaining its proper subcellular localization, KAI-9803 inhibits δPKC's physiological activity by inhibiting phosphorylation of specific protein substrates and by interrupting its endogenous subcellular localization.

Mechanistic studies have determined that a δPKC antagonist such as KAI-9803 inhibits a very early signaling event following ischemia and reperfusion by preventing δPKC subcellular localization to the mitochondria. Without desiring to be bound by theory, the inventors believe that these actions may enhance mitochondrial structural integrity and functionality and may mitigate myocardial cellular necrosis and apoptotic cell injury. Preservation of myocardial and endothelial cellular function is therefore believed to result in enhanced ATP generation and mitochondrial function and may improve compensatory contractile activity in healthy myocardial cells in the non-infarct zone.

Further, ex vivo animal studies have been carried out to demonstrate the efficacy of KAI-9803 in ameliorating reperfusion injury.

Hearts from adult male rats subjected to global ischemia and reperfusion were treated with KAI-9803 and demonstrated a significant recovery of left ventricular end diastolic pressure (LVEDP), end diastolic pressure (EDP), and aortic perfusion pressure (PP) as well as 70% less release of creatine kinase (CK) during the first 30 minutes after reperfusion compared with controls. Hearts treated with KAI-9803 demonstrated no changes in these parameters in the absence of ischemia and reperfusion.

The present invention provides PKC agonist or antagonist peptides, therapeutic peptide compositions and methods for treating diseases or conditions. In particular, the present invention provides a method is provided for treating acute STEMI. In a typical embodiment, the method comprises: administering to a patient suffering from STEMI a therapeutically effective amount of a PKC isozyme-specific antagonist, such as a δPKC antagonist.

In one embodiment of the present invention, a PKC isozyme-specific antagonist is used for the treatment of reperfusion injury in humans following an ischemic event, such as STEMI. In the clinical treatment to reduce tissue damage due to ischemia-reperfusion, the PKC antagonist peptides of the present invention may have the following attributes and advantages, compared with treatment using conventional therapeutics or techniques.
The immediate improvements of ejection fraction and regional wall motion. For example, KAI-9803 significantly improved ejection fraction and reduced wall motion deficits as early as 30 minutes after reperfusion. Typically, myocardial stunning and regional contractile dysfunction take up to five days to stabilize following STEMI.
The beneficial effects on coronary flow reserve and microvascular function. For example, as seen by electron microscopy, preservation of endothelial cell integrity with KAI-9803 limits occlusion of these small vessels. Furthermore, KAI-9803 showed immediate improvements in CFR measurements following reperfusion.
Significant pharmacological benefits at very low doses and thus a large therapeutic index with a favorable human safety profile.

In another embodiment of the present invention, a method is provided for preventing or treating tissue damages due to insufficient oxygen. The method comprises: administering to a human a therapeutically effective amount of a PKC isozyme-specific antagonist.

In another embodiment, a method is provided for reducing infarct size, limiting myocardial cellular damage, enhancing recovery of myocardial metabolic activity and/or regional left ventricular contractile function, and improving endothelial function in the infarct zone. Preferably, a δPKC antagonist is administered to the human subject. In one preferred embodiment, KAI-9803 is administered to a patient for ameliorating infarct size and improving myocardial contractile function following reperfusion with primary percutaneous coronary intervention (PCI).

Given that microvascular dysfunction following successful epicardial reperfusion is associated with larger infarct size, progressive left ventricular dilatation, and increased rates of mortality and congestive heart failure, adjunctive therapies designed to ameliorate reperfusion injury and improve microvascular function following epicardial reperfusion may be expected to improve myocardial salvage and thus result in improved left ventricular mechanical function and sustained clinical benefit.

Various routes of administration and dosing regimens may be used for administering a therapeutically effective amount of the PKC isozyme-specific antagonist to a human.

The PKC isozyme-specific antagonist may be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The PKC isozyme-specific antagonist may also be administered or coadministered in slow release dosage forms. Preferably, the PKC isozyme-specific antagonist is administered via local delivery to the heart, e.g., via intracoronary delivery. More preferably, the PKC isozyme-specific antagonist is administered via intracoronary delivery by a catheter.

The PKC isozyme-specific antagonist may be administered to a patient at risk of or suffering from tissue damage due to insufficient oxygen at any time as long as it is therapeutically effective to prevent or treat the tissue damage. For example, the PKC isozyme-specific antagonist can be administered 1-300 minutes, optionally 1-180 minutes, optionally 1-90 minutes, optionally 1-60 minutes, or optionally 30 minutes, before, during, or after the occurrence of oxygen-deficiency event, such as ischemia, anoxia, hypoxia, reperfusion and other environmental stresses.

The dose at which the PKC isozyme-specific antagonist may be administered to a human may vary, depending on the particular route of administration.

For example, for intracoronary administration, the PKC isozyme-specific antagonist may be administered at a low dose owing to its high specificity and selectivity for a particular PKC isozyme, e.g., at a dose of at least 0.0001 mg, optionally at least 0.001 mg, optionally at least 0.01 mg, optionally at least 0.05 mg, optionally at least 0.1 mg, optionally at least 0.5 mg, or optionally at least 1 mg.

Also, owing to its superior toxicity profile compared to conventional therapeutics, the PKC isozyme-specific antagonist may be administered at a higher dose via various routes of administration described above, e.g., at dose of 0.01-1000 mg, optionally at dose of 0.1-500 mg, optionally at dose of 1-200 mg, optionally at dose of 1-100 mg, optionally at dose of 1-50 mg, optionally at dose of 1-20 mg, optionally at dose of 0.1-5 mg, or optionally at dose of 1-5 mg.

Alternatively, the PKC isozyme-specific antagonist may be administered a human at a dose calculated based on the suffer area of the human, e.g., at dose of 0.01-1000 mg/m², optionally at dose of 0.1-500 mg/m², optionally at dose of 1-200 mg/m², optionally at dose of 1-100 mg/m², optionally at dose of 1-50 mg/m², optionally at dose of 1-20 mg/m², optionally at dose of 0.1-5 mg/m², or optionally at dose of 1-5 mg/m².

The treatment cycle may be once, twice per day, 1 or 2 weeks per month. In a preferred embodiment, the PKC isozyme-specific antagonist is administered intracoronarily first by direct bolus injection over approximately one minute, and then delivered by a guide catheter to the site of the body at risk of or suffering from tissue damages.

The PKC isozyme-specific antagonist may be in any dosage form suitable for a particular route of administration. For example, the PKC isozyme-specific antagonist may be delivered to a human in a form of solution that is made by reconstituting a solid form of the drug with liquid. This solution may be further diluted with infusion fluid such as 0.9% sodium chloride injection, 5% dextrose injection and lactated ringer's injection. It is preferred that the reconstituted and diluted solutions be used within 4-6 hours for delivery of maximum potency. Alternatively, the PKC isozyme-specific antagonist may be delivered to a human in a form of tablet or capsule.

In some embodiments, the methods of the present invention may further include a step of evaluating treatment success (or the degree thereof) based on the measurement of various biomarkers. In other embodiments, the methods of the present invention may further include a step of identifying a patient in need of treatment based on the measurement of various biomarkers.

Resolution of ST-segment elevation is a non-invasive biomarker that correlates with reperfusion of the infarct-related artery (IRA), successful restoration of myocardial tissue perfusion, and a lower risk of mortality (Schroder et al. (1994) J Am Coll Cardiol 24:384-91; Santoro et al. (1998) J Am Coll Cardiol 82:932-7; and de Lemos (2001) J Am Coll Cardiol 38:1283-94). Continuous analysis of ST-segment recovery characterizes the entire process of reperfusion, and ST-recovery parameters correlate directly with preservation of left ventricular mechanical function, early reinfarction, and mortality (Andrew et al. (2000) Circulation 101:2138-43; Moons et al. (1999) Am. Heart J 138: 525-32; Langer et al. (1998) J Am Coll Cardiol 31:783-8; Shah et al. (2000) J Am Coll Cardiol 35:666-72). Acute angiographic measurements of treatment success have also correlated with clinical outcomes and can be used to directly visualize epicardial and microvascular flow as well as myocardial tissue perfusion (Gibson et al. (1999) Circulation 99:1945-50; and Gibson (2000) Circulation 101:125-30). Finally, assessment of serial measurements of early cardiac marker release patterns such as creatine kinase (CK) or CK-MB (myocardial band) has proven useful for evaluating early infarct size and for characterizing reperfusion success for patients treated with fibrinolytic therapy (Vollmer et al. (1993) Am. J. Clin. Pathol. 100:293-298; Christenson et al. (2000) J Am Coll Cardiol 85:543-7; van der Laarse et al. (1988) Am. Heart J 115:711-6; and Baardman et al. (1996) Eur. Heart J 17:237-246).

Other techniques for evaluating reperfusion success (or the degree thereof) involved delayed imaging after initial reperfusion to assess myocardial contractile recovery and remodeling, infarct size, and residual myocardial viability in the infarct zone. Cumulative infarct-size measurement with ^{99m} Tc-sestamibi SPECT may be used to determine the degree of left ventricular damage and the amount of myocardial salvage after treatment (Miller et al. (1995) Circulation 92:334-41; and Gibbons et al. (2000) Circulation 101:101-8). Echocardiography also appears to be useful for evaluating regional wall motion and ventricular remodeling after STEMI (Solomon et al (2001) Ann Int Med 134:451-8; and Aikawa et al. (2001) Am Heart J 141:234-42). Finally, cardiac magnetic resonance imaging (MRI) may be the most comprehensive imaging technique following STEMI because it can be used to assess coronary flow, myocardial tissue perfusion, left ventricular volumes, and regional and global left ventricular function (Wu et al. (1998) Circulation 97:765-72; and Bremerich et al. (1998) J Am Coll Cardiol 32:787-93).

Also according to the present invention, the methods of the present invention may further include a step of evaluating reperfusion success (or the degree thereof) based on the measurements of an array of biomarkers.

Use of a single biomarker alone may be a limited approach to evaluating promising new reperfusion regimens. Missing or un-interpretable data from individual biomarkers limits the predictive capabilities of a single biomarker. Furthermore, correlations between individual biomarkers may be used for evaluating new therapies, but the relative utility of single biomarkers for comprehensively describing the speed and quality of reperfusion remains unclear. Studies that have used multiple, simultaneous biomarkers suggest that biomarker arrays may be the most effective approach for the evaluation of new therapies for STEMI, both by enhancing information content and retrieval and by overcoming the loss of data that occurs when individual biomarkers are used (23-25). The therapeutic success of new reperfusion regimens has not yet been tested with biomarker arrays. Nonetheless, biomarker arrays may be used to assess the potential efficacy of promising new reperfusion regimens and adjunctive therapies for STEMI.

### EXAMPLES

### Example 1

### Exopeptidase Protection: Plasma Stability of Capped Peptides

Plasma stability of capped peptides was compared. KAI-9706 was modified step-wise at its amino and carboxy termini. Plasma stability as measured by the percent of peptide composition remaining after 15 minutes. The results are provided in Table 3.

TABLE 3
Plasma Stability of KAI-9706

| | | cargo | | | |
|---|---|---|---|---|---|
| | | H—OH | Ac—OH | H—NH₂ | Ac—NH₂ |
| carrier | H—OH | 1 | 1 | 0 | 0 |
| | Ac—OH | 57 | nd | nd | 48 |
| | H—NH₂ | 60 | nd | nd | 51 |
| | Ac—NH₂ | 92 | 93 | 90 | 90 |

% parent remaining at 15 mins
t_{1/2} in rat plasma = 40-45 mins for longest-lived derivatives

The data provided above shows that the peptide composition, comprising unmodified cargo and carrier peptides, was the least stable. Moreover, protection of the carrier peptide alone failed to increase the half life of the peptide composition in plasma. Moreover, modification of the cargo peptide with the carrier peptide unmodified had no apparent effect on half-life stability in plasma. However, the addition of protecting groups to the carrier peptide, whether at the amino or carboxy termini lead to a marked and nearly equivalent increase in plasma stability for the peptide composition. Protection of both groups in the carrier peptide provided additional protection. Interestingly, protection of the cargo peptide had little or no effect on the stability of the composition.

### Example 2

### Effect of d-Peptides on Plasma Stability

KAI-9706 was engineered with D-amino acids to determine their impact on peptide composition stability. Unmodified KAI-9706 was compared to a peptide composition with the same amino acid sequence, however the amino acids used were d-enantiomers instead of 1-amino acids. A retro-inverso version and a scrambled version of the peptide composition were also prepared. The data from the experiment is shown in Table 4.

TABLE 4
Plasma Stability of KAI-9706

cargo

| | | All-L | All-D | scrambled | R/I |
|---|---|---|---|---|---|
| carrier | All-L | 1 | 0 | 2 | |
| | All-D | 88 | 100 | 67 | |
| | R/I | | | | 100 |

% parent remaining at 15 mins

Modification of the carrier showed the great propensity in improving the half life of the composition while modification of the cargo showed little effect.

### Example 3

### Capped KAI-9706 Maintains In Vitro Potency

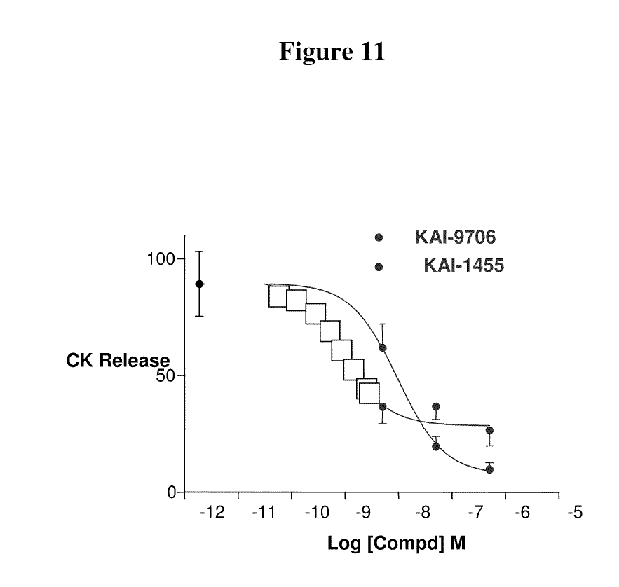

Capping the carrier peptide portion KAI-9706 (KAI-1455) was shown to increase the plasma half life of the peptide composition. The ability of the capped composition to inhibit ischemic damage in a rat heat model (Langendorff Assay) was evaluated versus the uncapped form. The results are shown in FIG. 11.

### Example 4

### Capped KAI-9706 Shows Increased Potency

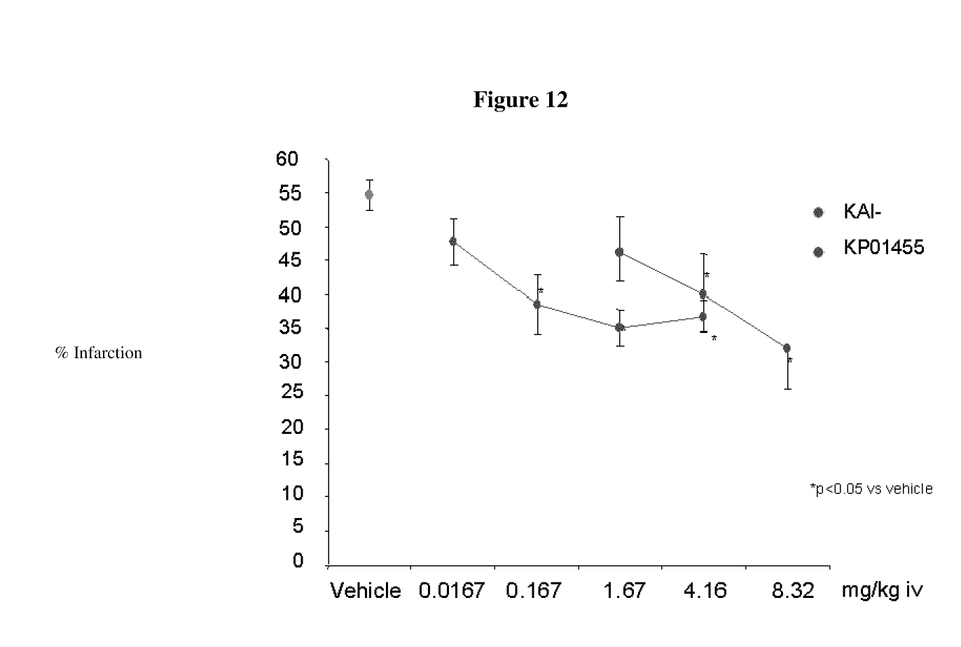

KAI-1455 was tested in a stroke model. As shown in FIG. 12, the capped version of the peptide composition provided increased protection to brain tissue as judged by percent infract. This data shows that significant protection of brain tissue was achieved at lower doses.

### Example 5

### Peptide Stability is Increased Regardless of Species

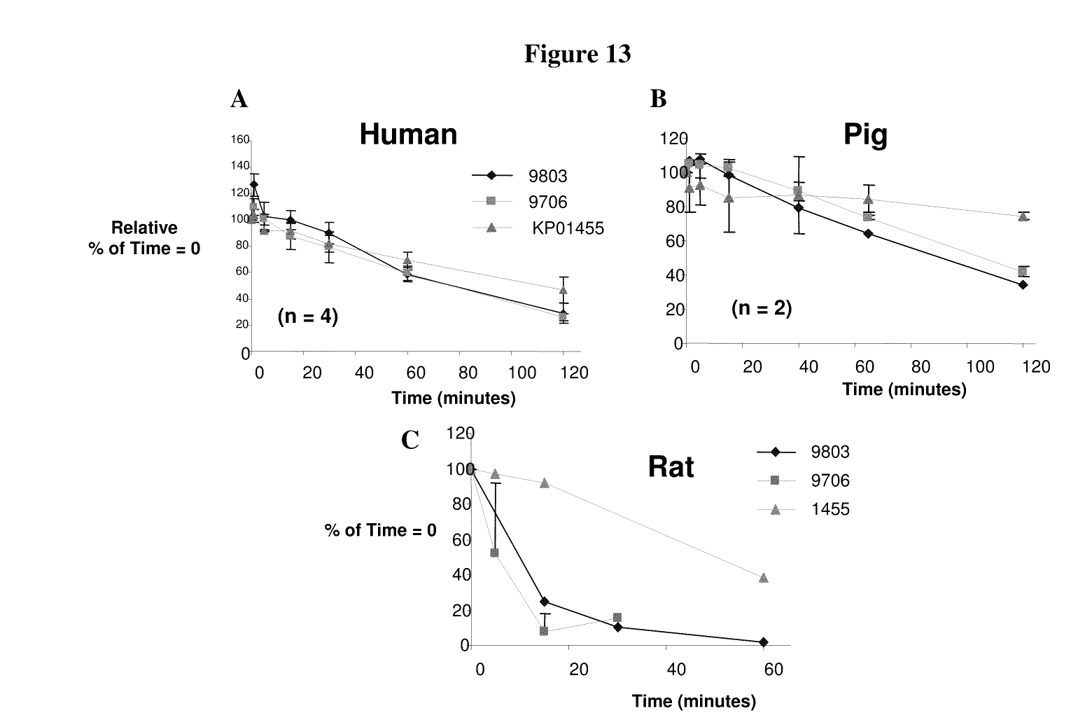

The stability of modified KAI-9706 peptide (KAI-1455) was compared against KAI-9706 and KAI-9803 in human (FIG. 13A), pig (FIG. 13B) and rat serum (FIG. 13C). The capped version, KAI-1455, showed increased plasma stability in all three species.

### Example 6

### Capped KAI-9706 Shows Increased Potency

KAI-1455 was tested in a stroke model. As shown in FIG. 12, the capped version of the peptide composition provided increased protection to brain tissue as judged by percent infract. This data shows that significant protection of brain tissue was achieved at lower doses.

### Example 7

### Peptide Stability is Increased Regardless of Species

The stability of modified KAI-9706 peptide (KAI-1455) was compared against KAI-9706 and KAI-9803 in human (FIG. 13A), pig (FIG. 13B) and rat serum (FIG. 13C). The capped version, KAI-1455, showed increased plasma stability in all three species.

### Example 8

### Stability of Linear Peptides

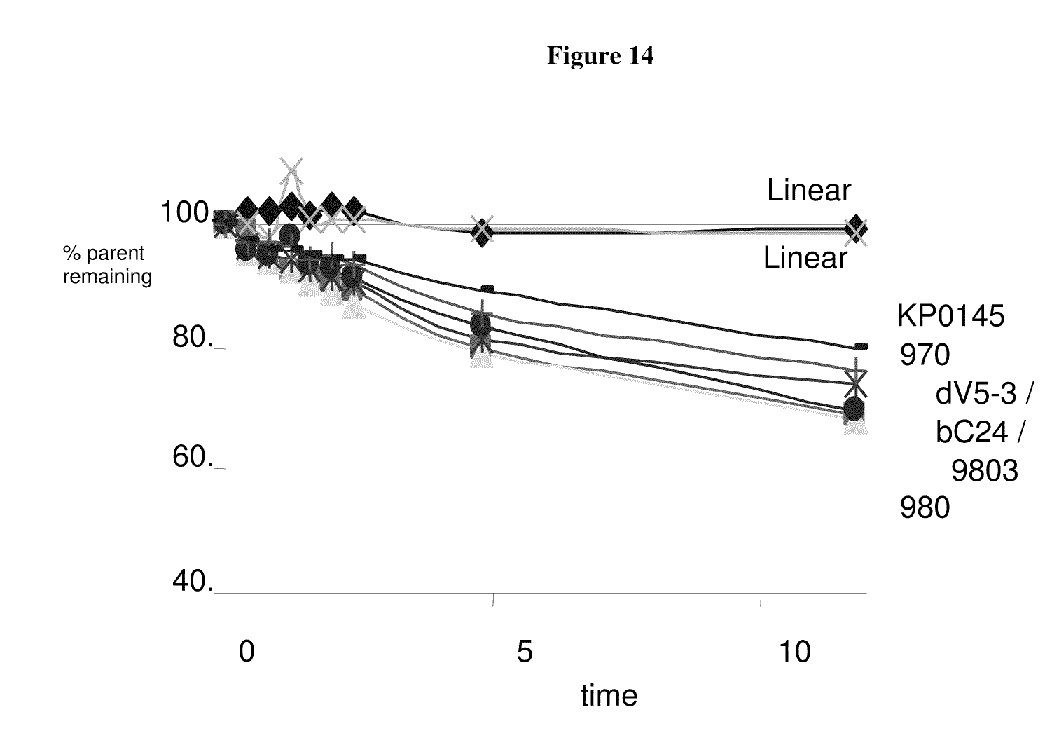

Linear versions of KAI-9803 and BC2-4 were constructed to evaluate their stability relative to disulfide bond linked versions of these and other peptide compositions. The peptides were placed in solution at 0.1 mg/ml in PBS (pH 7.4) at 37° C. As shown in FIG. 14, the linear versions of KAI-9803 and BV2-4 showed increased stability.

### Example 9

### Linear PKC-β_{I} and PKC-β_{II} Peptide Compositions Show Increased Stability Over Disulfide Linked Compositions

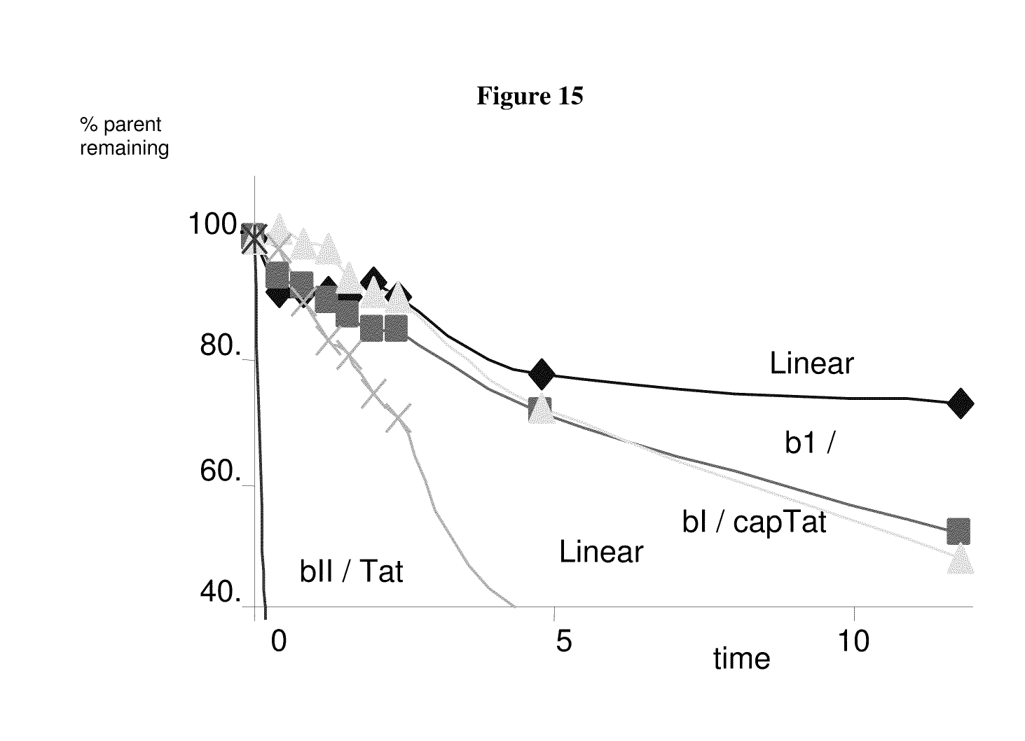

Linear and disulfide bond linked versions of PKC-β_{I} and PKC-β_{II} peptide compositions were incubated under the conditions described in Example 15. As can be seen in FIG. 15, the linear forms of the PKC-β_{I} and PKC-β_{II} peptides showed increased stability as compared to their non-linear counterparts.

### Example 10

### Improved Stability of Linear PKC-β_{I} and PKC-β_{II} Peptide Compositions

The linear versions of PKC-β_{I} and PKC-β_{II} peptide compositions showed improved stability but were also the subject of deamination reactions. In particular, the Asn residues at position 7 of the β-I and β-II peptides and the Gln at position 2 of the β-II peptide. These linear peptide compositions were modified by substituting the Gly immediately C-terminal to the Asn with either Leu in the β-I peptide composition or Gly to Ile in the β-II peptide composition. The Gln at position 2 of the β-II peptide composition was also substituted with a Glu residue. The stability of the peptides was studied under the conditions described in Example 15. As shown in FIGS. 8 and 9, the amino acid substitutions discussed above served to stabilize these linear peptide compositions.

### Example 11

### KAI-9803 Derivative (KAI-1355) Maintains Potency

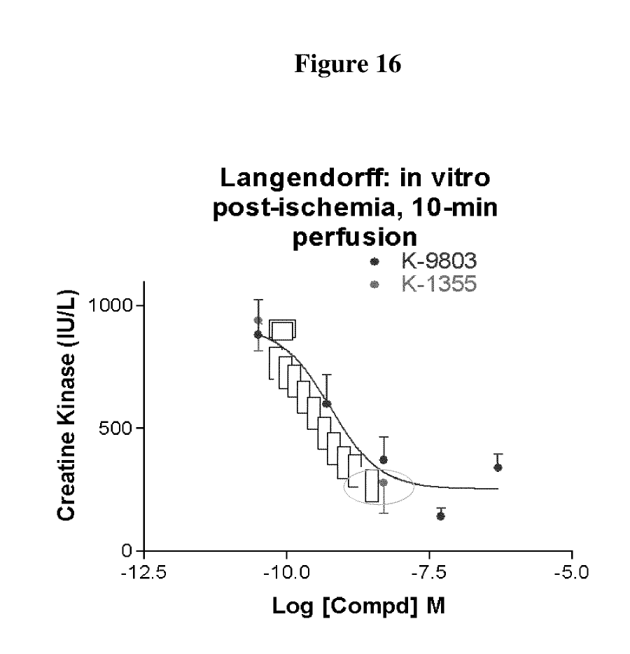

A truncated version of KAI-9803, KAI-1355, in which the carboxy terminal leucine was removed was tested for potency. Stability studies with KAI-1355 showed that deletion of the C-terminal Leu residue increased the stability of this cargo peptide. Potency of the derivative peptide composition was compared to that of the full length version, KAI-9803 in a Langendorff in vitro post-ischemia model. The results of the experiment are shown in FIG. 16. As shown, KAI-1355 (the modified version of KAI-9803) is still capable of protecting cardiac tissue from ischemia with potency comparable to that of the full length KAI-9803.

### Example 12

### Optimization of KAI-9803 to Produce KAI-1479

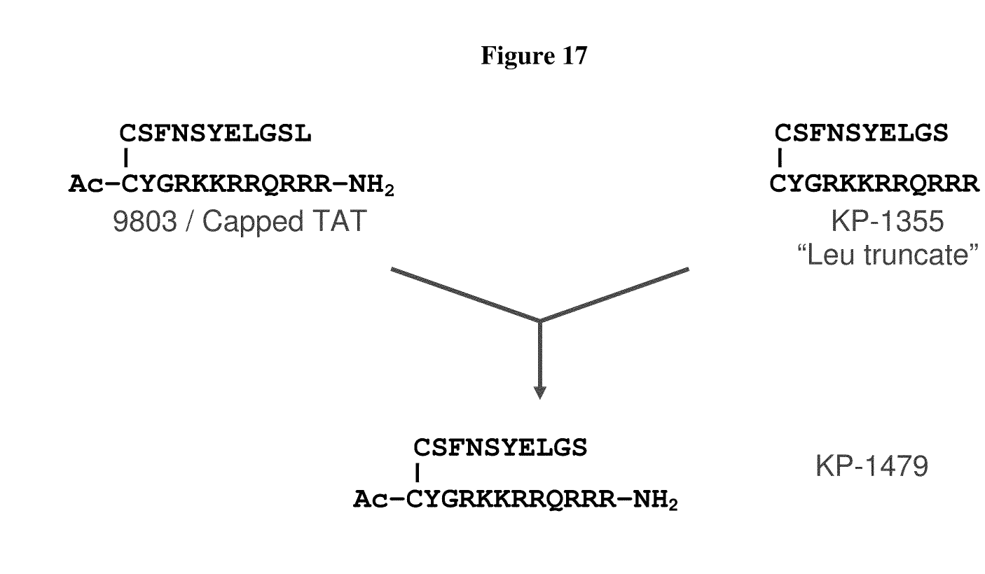

Having demonstrated that truncation of the cargo peptide of KAI-9803 had little or now effect on potency, while stabilizing the peptide composition. As illustrated in FIG. 17, a capped version of the TAT carrier peptide was bound to the truncated cargo peptide of KAI-1355 (CSFNSYELGS) (SEQ ID NO:62), producing KAI-1479, which comprises a truncated 9803 cargo peptide and a fully capped TAT carrier peptide.

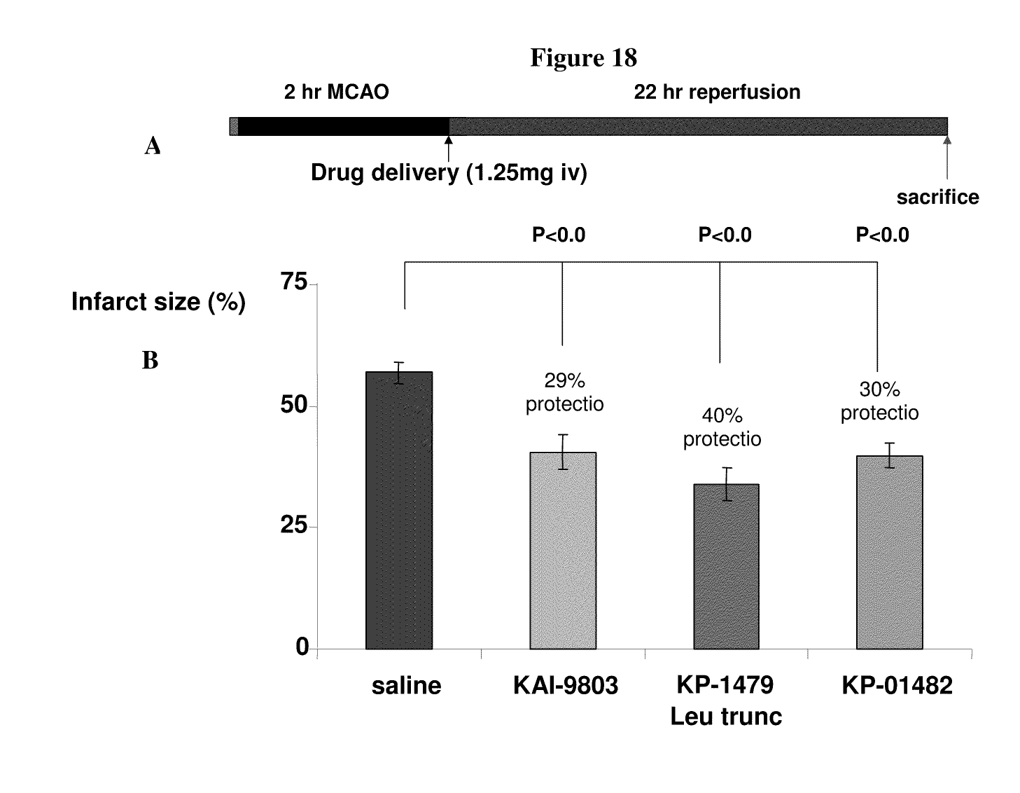

The modified KAI-1479, KAI-9803 and KAI-1482 peptide compositions were assayed in a rat middle cerebral artery occlusion (MCAO) stroke model to determine the ability of the peptide compositions to inhibit infarct size. The rats were subjected to a 2 hour period of cerebral arterial occlusion. Each of the peptide compositions or saline was administered to the test animals immediately prior to a 22 hour reperfusion period, after which time the animals were sacrificed and the infarct size was measured. As shown in FIG. 18, the modified KAI-1479 peptide composition showed an increased ability to retard infarct size as compared to KAI-9803. KP-01482 has a cargo sequence (CELGSLQAEDD) (SEQ ID NO:63) linked to a TAT peptide with a N-Term Cys, which is capped at both ends and disulfide conjugated to the cargo. KP-1479 is based off of KP-1226, which has the same components but is not capped.

### Example 13

### In Vitro Biological Stability of a Series of Linear Epsilon PKC Inhibitors

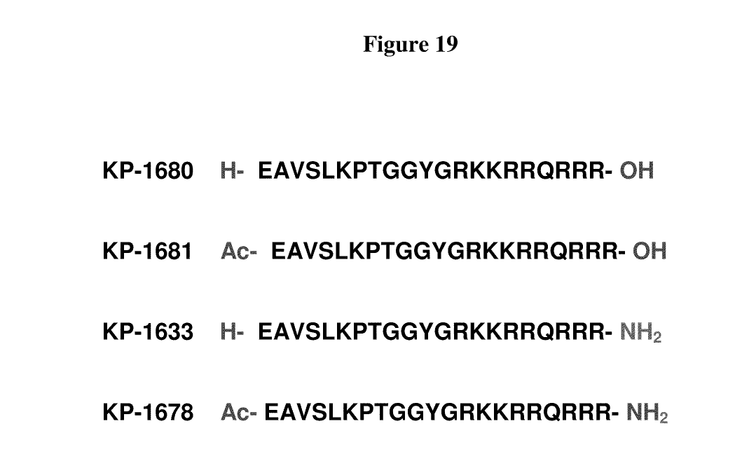

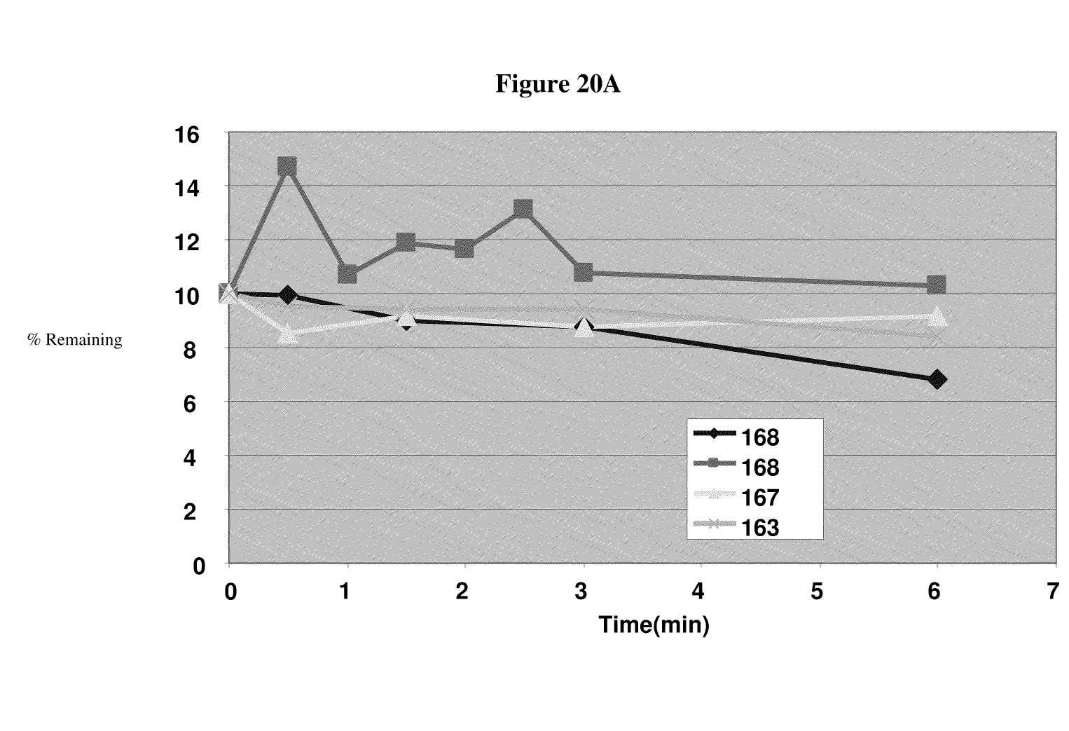

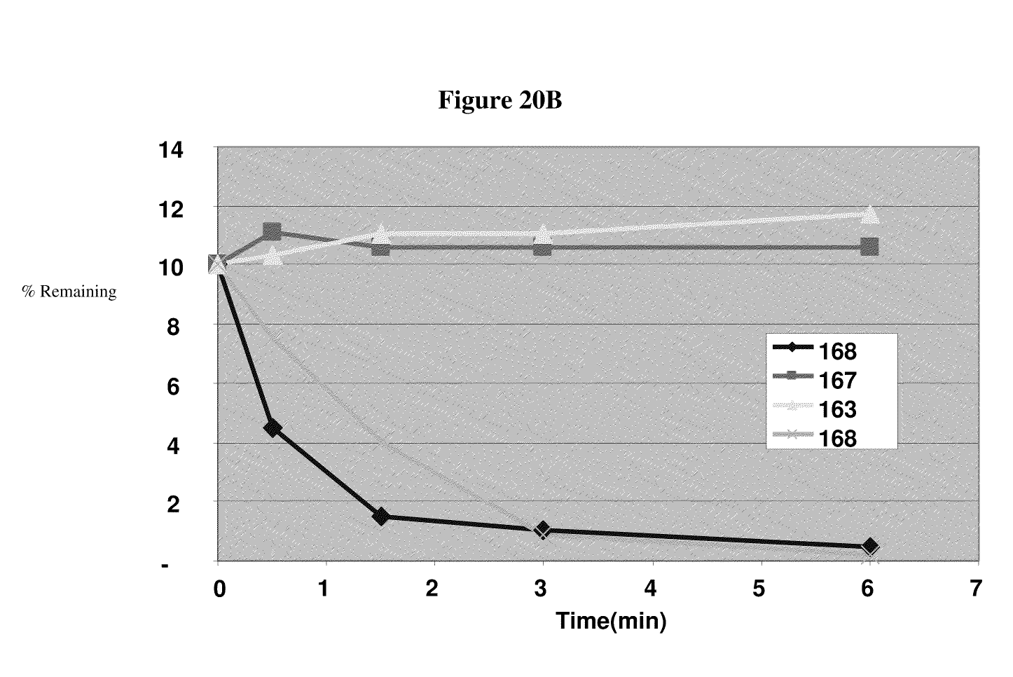

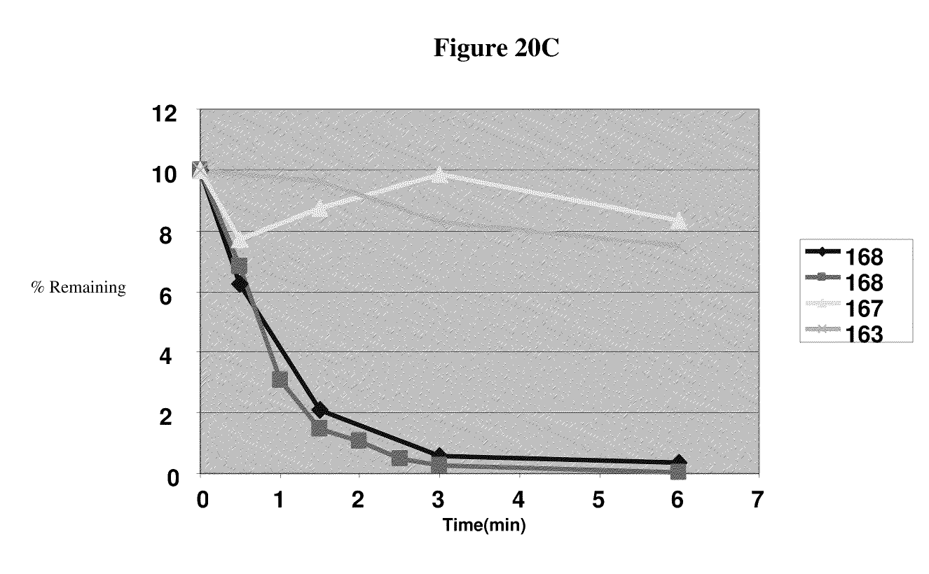

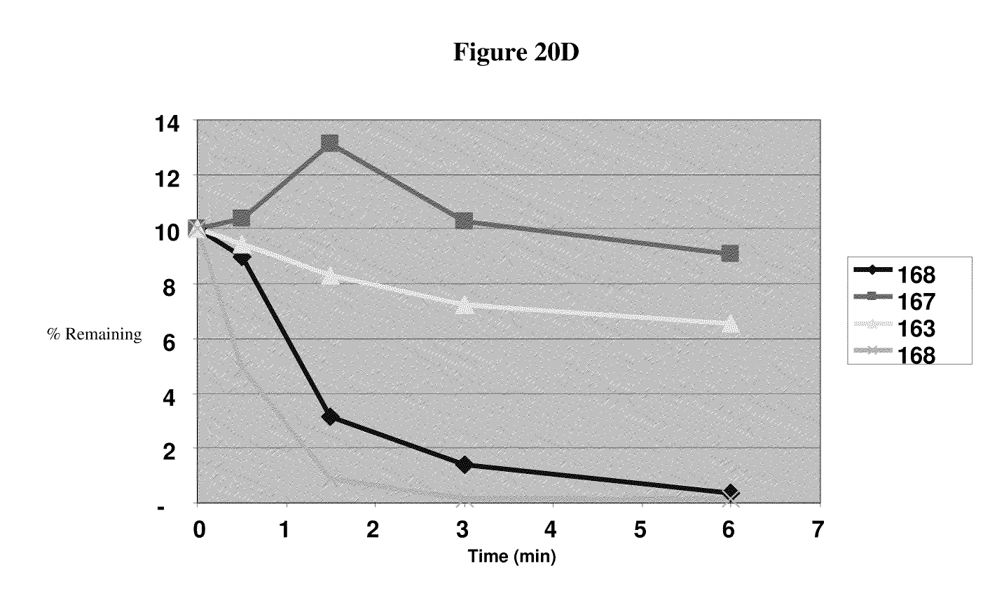

The effect of N-terminal acetylation and C-terminal amidation on compound stability in plasma and serum from rat and human was studied. The linear peptides examined are shown in FIG. 19. The compounds were tested in the plasma/serum at a concentration of 100 μg/ml. The solutions were incubated at room temperature, precipitated with 5% TCA, and then the supernatant was neutralized with ammonium acetate. The peptides were then analyzed by LC/MS. As can be seen from the data in FIGS. 20A-20D, all the tested compounds were relatively stable in human plasma but KP-1633 and KP-1678, containing C-terminal amides, showed increased stability in human serum. N-terminal acetylation alone did not stabilize the peptides. Interestingly, the amino acid sequence of KP-1680 and its degradation products indicated that the metabolized forms of the peptide showed sequential cleavage of arginine residues from the C-terminus. Carboxypeptidase N activity in the serum but not the plasma could account for the difference in observed stability. The plasma samples were collected with EDTA, which is known to inhibit this zinc metalloprotease.

### Example 14

The following example is a clinical treatment of acute ST-elevation myocardial infarction (STEMI) using the compositions and methods provided in the invention. Specifically, various doses of KAI-9803 are administered to humans via intracoronary injection during primary percutaneous coronary intervention (PCI) for STEMI. The effect of KAI-9803 on multiple biomarkers that reflect myocardial viability and myocardial tissue perfusion are assessed both as a measure of safety and to evaluate the drug efficacy.

### Patient Inclusion Criteria and Consent

Patients with STEMI expected to undergo primary PCI were enrolled if they were >18 years of age, presented with at least 30 minutes of ischemic chest pain and within 6 hours of symptom onset, had persistent ST-segment elevation of ≧0.2 mV in at least 2 contiguous precordial leads indicating anterior MI location (leads V1-V4), had complete occlusion of the left anterior descending artery (TIMI 0-1 flow) on the initial diagnostic angiogram, and had a culprit lesion suitable for primary PCI.

Key exclusion criteria included: left bundle branch block (new or old), intraventricular conduction defect, prior documented MI including old Q waves on prior electrocardiograms (ECGs) or a clinical history of definite MI, prior coronary artery bypass grafting (CABG), cardiogenic shock at initial hospital presentation, treatment with intravenous fibrinolytic therapy within 24 hours before enrollment, and known baseline serum creatinine >2.5 mg/dL without renal dialysis/renal replacement therapy within 30 days of randomization.

All patients provided written informed consent, and the protocol was approved by the institutional review board of each participating institution. Patients were consented before the start of the diagnostic angiogram.

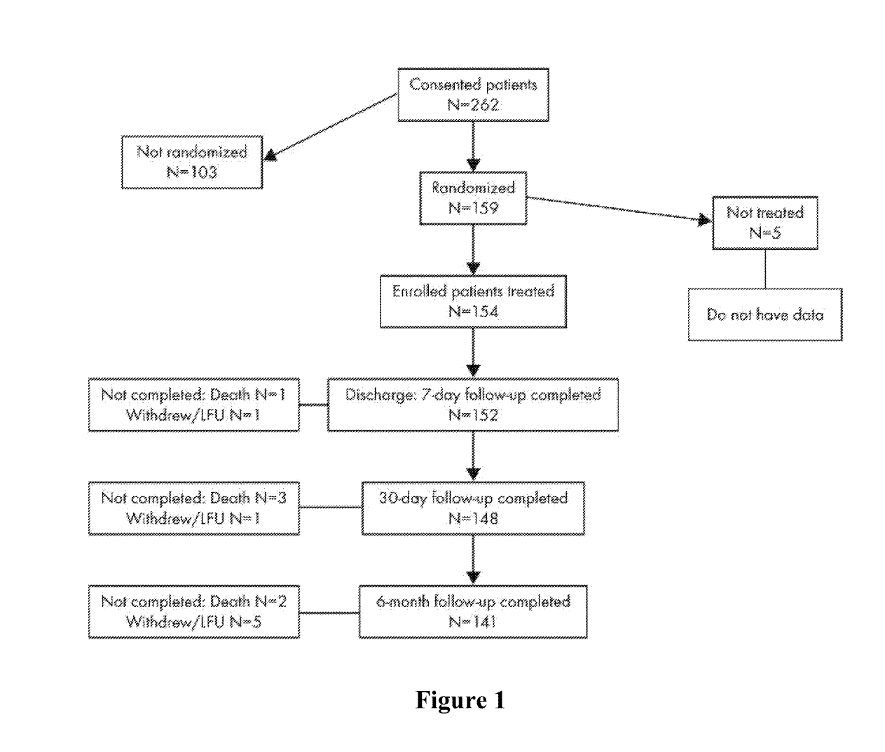

From September 2004 through May 2006, a total of 262 patients were consented for inclusion in the study, and 159 patients were randomized (FIG. 1). The most common reasons for patients not being eligible for randomization after informed consent was obtained included TIMI 2-3 flow in the LAD on the initial diagnostic angiogram (n=85), non-angiographic inclusion criteria were not met (n=9), and need for urgent bypass surgery (n=2). Of the 159 randomized patients, 5 patients were not treated with study drug due to inability to cross the culprit lesion during PCI (n=2) or other technical reasons related to drug preparation (n=3). Thus, the modified intent-to-treat population (which formed the primary analysis population) included 154 patients who were randomized and treated with some amount of study drug. Among these patients, 37 were treated in cohort 1, 38 in cohort 2, 38 in cohort 3, and 41 in cohort 4.

The majority of patients enrolled in cohorts 1 and 2 were from the United States and Canada, whereas the majority of patients enrolled in cohorts 3 and 4 were from Europe and Brazil. The median patient age ranged from 55-64 years, approximately 70-80% of the patients were male, and the proportion of patients with cardiac risk factors such as diabetes mellitus varied across dosing cohorts. In all dosing cohorts, a higher proportion of patients receiving active study drug presented in Killip class II or III compared with concurrent placebo. The median time from symptom onset to randomization increased progressively across dosing cohorts and was between 171-174 minutes in cohort 1 compared with 234-256 minutes in cohort 4. The maximum degree of ST elevation before PCI also increased progressively across dosing cohorts, ranging from 522-700 uV. (See Table 1)

### Treatment and Randomization Procedures

Patients were recommended to receive aspirin (162-325 mg orally), a thienopyridine loading dose (clopidogrel 300-600 mg or ticlopidine 500 mg), unfractionated heparin, and glycoprotein IIb-IIIa inhibitors during PCI. Within each dosing cohort, patients were randomly assigned 2:1 to receive active study drug (KAI-9803) or saline placebo. After verifying the final angiographic inclusion criteria during diagnostic angiography, randomization and study drug preparation occurred in the catheterization laboratory before the beginning of the PCI procedure.

Each study drug kit contained either a blinded 20 mL glass vial containing 5 mg of KAI-9803 or a blinded empty 20 mL vial. KAI-9803 was formulated as a sterile powder containing 5 mg of KAI-9803 and 40 mg of mannitol in a lyophilized formulation. Study drug was reconstituted into a clear solution at 1 of 4 concentrations using serial dilutions with 0.9% sodium chloride solution. Study drug was administered during PCI in the following sequence. First, the guidewire was passed across the obstruction in the left anterior descending (LAD) infarct vessel and an over-the-wire balloon catheter was positioned at the level of the obstruction. Second, the guidewire was removed, and dilute contrast was injected to confirm positioning of the catheter tip downstream from the obstruction and to assess patency of the distal vessel. The contrast injection was followed by a saline flush, and the guidewire was reinserted. Third, the balloon was inflated, and, after removing the guidewire, 2 mL of blinded study drug were hand injected slowly into the distal vascular bed over at least 1 minute. Fourth, the guidewire was readvanced through the catheter into the distal vessel, and the balloon was deflated. Finally, after antegrade epicardial coronary flow was re-established following initial balloon inflations, 3 mL of blinded study drug were injected through the guide catheter positioned at the ostium of the left main coronary artery. The PCI procedure was then completed according to standard technique.

Ascending dosing cohorts were designed to sequentially evaluate progressively higher doses of KAI-9803. The total dose of KAI-9803 administered in cohort 1 was 0.05 mg (0.02 mg with first dose, 0.03 mg with second dose) and subsequently total doses of 0.5 mg (0.2 mg with first dose, 0.3 mg with second dose), 1.25 mg (0.5 mg with first dose, 0.75 mg with second dose), and 5.0 mg (2.0 mg with first dose, 3.0 mg with second dose) were studied in cohorts 2, 3, and 4, respectively. The first dose delivered via balloon catheter in cohort 1 (0.02 mg) corresponds to the lowest efficacious dose tested in preclinical studies when adjusted for the increased cardiac mass in humans.

The median total ischemia time (time from symptom onset to re-establishment of flow with PCI) increased progressively across dosing cohorts. Median total ischemia times were longer in the active study drug groups compared with concurrent placebo groups in cohorts 3 (299 vs. 211 minutes) and 4 (286 vs. 248 minutes), respectively. The culprit lesion was located in the proximal LAD in approximately one third of patients, and the proportion of patients with significant visible collaterals to the LAD distribution varied from 23-62%. Glycoprotein IIb-IIIa inhibitors were administered to the majority of patients during PCI. Antegrade flow was successfully re-established in all patients per the investigators' reports, and all patients received both doses of study drug. Post-PCI corrected TIMI frame count results were similar across dosing cohorts while the proportion of patients with normal TIMI myocardial perfusion grade 3 was non-significantly higher with active study drug compared with placebo in cohorts 2 and 3. Coronary flow reserve measurements were similar among patients treated with active study drug vs. those treated with concurrent placebo in each dosing cohort. (See Table 2.)

### Collection of Biomarker End Points

After initial informed consent was obtained and before diagnostic angiography was performed, a continuous digital 12-lead ECG monitor (NEMON 180+, Northeast Monitoring, Natick, Mass.) was applied and baseline serum samples were obtained for analyses of cardiac markers including creatine kinase (CK and CK-MB), troponin T, and N-terminal pro-brain natriuretic peptide (BNP). These data were destroyed for patients who were consented but not randomized.

After completion of the PCI procedure, angiography was performed a minimum of 10 minutes after the final balloon inflation and a minimum of 30 minutes after antegrade epicardial flow was re-established to measure TIMI (Thrombolysis in Myocardial Infarction) flow grade, corrected TIMI frame count, and TIMI myocardial perfusion grade (TMPG). TMPG was assessed approximately 30 minutes after administration of study drug and prior to intracoronary administration of adenosine. Coronary flow reserve was determined by measuring corrected TIMI frame count before and after administration of 18-24 mcg of intracoronary adenosine. A left ventriculogram was performed to assess ejection fraction.

The continuous ECG monitor was removed after 24 hours of data collection. Continuous ST-segment recovery analysis was performed in a blinded core laboratory. End points assessed from measurement of continuous 12-lead ECGs included time to stable ST recovery, % ST-segment resolution at serial static time points, and ST recovery time-trend curve area defined by summated ST-segment resolution and re-elevation integrated over the first 3 hours following PCI.

Blood collection for CK-MB levels was performed at 3-6 hours, 6-12 hours, 18-24 hours, and 36 hours after randomization. CK-MB end points assessed included infarct size by CK-MB area-under-the-curve (AUC) and estimated peak CK-MB values as determined through curve-fitting techniques. Additionally, N-terminal pro-BNP levels were drawn at 24 hours and on the day of discharge.

Patients were scheduled for a follow-up visit 14 days after randomization for blood collection for N-terminal pro-BNP levels, myocardial SPECT imaging using Technetium Tc-99m Sestamibi for final infarct size measurement, and transthoracic echocardiography for assessment of left ventricular ejection fraction and regional wall motion contractile function.

All core laboratories for the biomarker analyses were blinded to treatment assignment.

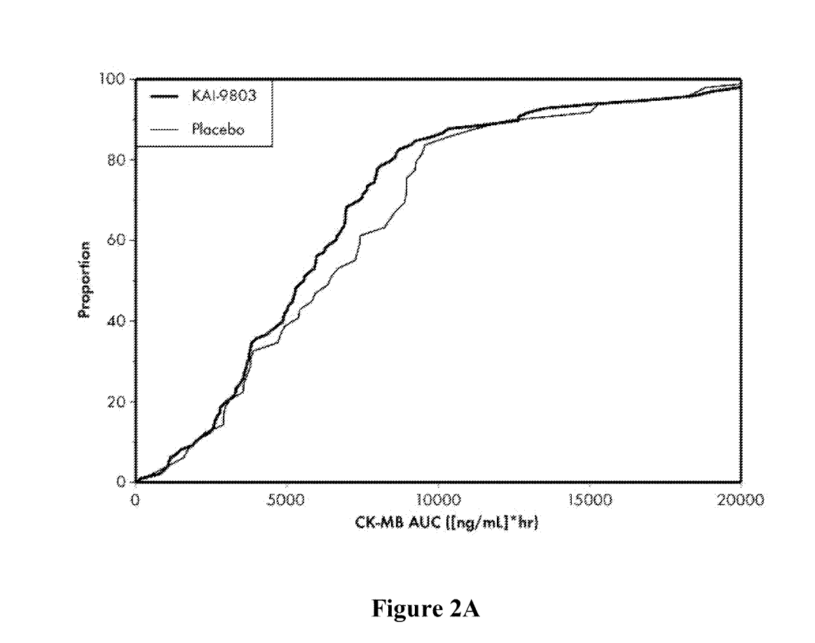

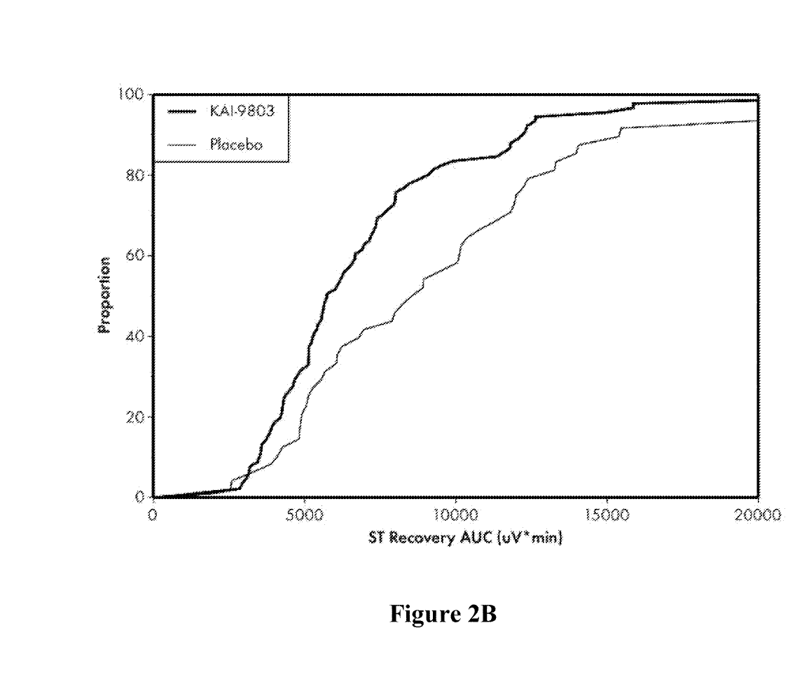

The median percentage of ST resolution at 60 minutes varied from 59-71% and was nonsignificantly higher with active study drug compared with concurrent placebo in each cohort, while the time to stable ST recovery was non-significantly more rapid with active study drug in cohorts 2 and 3. (Table 4.) Integrating both the extent and speed of ST recovery, ST-segment AUC values were non-significantly lower with active study drug in each cohort, except for the fourth dosing cohort where the difference was significant (P=0.04). Among the pooled active study drug and placebo populations, the median ST recovery AUC values were 8727 uV/min for placebo vs. 5735 uV/min for active study drug (P=0.005), while the cumulative distribution frequencies demonstrated a separation of ST-recovery AUC curves among patients with higher values (FIG. 2a).

Estimated peak CK-MB values and CK-MB (AUC) values were non-significantly lower with active study drug in each dosing cohort. Among the pooled active study drug and placebo populations, the median CK-MB AUC values were 6463 ng/ml*hrs for placebo vs. 5571 ng/ml*hrs for active study drug (P=0.27), while the cumulative distribution frequencies demonstrated a separation of CK-MB AUC curves among patients with values between 5000-10,000 ng/ml*hrs (FIG. 2b).

Infarct size values determined with Technetium Tc-99m Sestamibi were non-significantly lower with active study drug compared with concurrent placebo for cohorts 1, 2, and 3 and nonsignificantly higher with active study drug in cohort 4. Ejection fraction and LAD-specific wall motion score values were similar between active study drug vs. concurrent placebo within each cohort. Among the pooled active study drug and placebo populations, the median infarct size values were 31.5% for placebo vs. 30.0% for active study drug (P=0.96) when all available infarct size data were analyzed. When infarct size values collected only during the protocolspecified time window (10-35 days) were analyzed, the median infarct size values were 33% for pooled placebo vs. 26% for pooled active study drug populations.

### Safety End Points

Adverse events were assessed through hospital discharge or 7 days, whichever occurred first. Serious adverse events were assessed through 30 days after treatment. All adverse events were coded to a common dictionary (MedDRA). Investigators were asked to monitor for unexpected cardiopulmonary symptoms, arrhythmias, hemodynamic deterioration, and allergic symptoms during the initial PCI procedure and administration of study drug; arrhythmias (other than those that occur during reperfusion) requiring intervention; clinical end points as detailed below; ECG abnormalities such as QT interval prolongation, AV block, or symptomatic bradycardia requiring intervention; and ischemic or hemorrhagic stroke. Continuous ECG monitoring was used to assess QT intervals before, during, and after study drug administration during PCI. Standard laboratory values were assessed at baseline and at 24 hours and 48 hours after randomization.

No serious adverse events occurred that required early termination of study drug during the PCI procedure for any given patient or modification of the protocol based upon ongoing safety analyses by the DMC. Among the pooled populations, the median maximum change in the QT interval from baseline until after the second dose of study drug was 16.9 msec for patients treated with active study drug vs. 15.3 msec for those treated with placebo (P=0.83). Assessment of changes in clinical laboratory values, including complete blood counts, white blood cell count differentials, electrolytes, blood urea nitrogen, creatinine, and liver function tests from baseline through 48 hours, demonstrated no differences between active study drug vs. placebo. The incidence of serious adverse events through 30 days was 15.4% for pooled placebo and varied across active study drug cohorts (cohort 1, 30.4%; cohort 2, 16.0%; cohort 3, 19.2%; and cohort 4, 35.7%), but no significant differences were demonstrated (P=0.13 for trends across the active study drug groups compared with placebo). The majority of serious adverse events were cardiac disorders that were also captured as clinical events such as CHF, reinfarction, and cardiogenic shock. (Table 3.)

### Clinical End Points

Investigator-reported clinical outcomes were assessed through 6 months and were not adjudicated by an independent clinical events committee. End points included all-cause mortality, reinfarction, congestive heart failure (CHF), and infarct artery revascularization procedures (PCI and CABG). Reinfarction within 18 hours of randomization was defined as recurrent ischemic discomfort at rest persisting for at least 30 minutes accompanied by new or recurrent ST-segment elevation of ±0.1 mV in at least 2 contiguous leads. Reinfarction >18 hours after randomization was defined by a similar recurrence of ischemic discomfort with new Q waves in 2 or more leads or new left bundle branch block associated with re-elevation of CKMB levels above the upper limit of normal and increased by at least 50% over the most recent value before the recurrent ischemic event. Congestive heart failure at any time point after randomization was defined as any 1 of the following 3 scenarios: 1) cardiogenic shock with systolic blood pressure <90 mm Hg for more than 1 hour and signs of hypoperfusion; 2) physician's decision to treat signs or symptoms of CHF with an intravenous diuretic, intravenous inotropic agent, or intravenous vasodilator; or 3) evidence of CHF, including pulmonary edema on chest x-ray, rales >1/3 up lung fields, or pulmonary capillary wedge pressure >18 mm Hg measured with a pulmonary artery catheter.

Among the entire population, there were 6 deaths and 28 investigator-reported CHF events through 6 months. (See Table 6.) A total of 4 deaths and 24 CHF events occurred during the initial hospitalization. A total of 9 of the 21 CHF events in the active study drug group occurred in patients who had CHF on presentation (Killip class II or III), whereas no patients with CHF events in the placebo group had CHF on presentation. However, 12% of the pooled active study drug patients presented in Killip class II or III vs. 4% in the pooled placebo group. The 6-month mortality rates in the pooled active study drug and placebo groups were 3% vs. 6%, respectively.

### Study Design and Statistical Considerations

This first-in-human study was designed primarily to assess the safety of escalating doses of KAI-9803. This study was not designed or specifically powered to assess safety or efficacy by clinical or biomarker end points, but included a large enough sample of patients to provide an assessment of the safety and activity of KAI-9803 compared with saline placebo. A primary biomarker end point was not prespecified, but an array of biomarkers was evaluated to assess potential drug activity.

An independent Data Monitoring Committee (DMC) reviewed safety data through hospital discharge for the first 10 patients receiving a given dose of the active study drug (KAI-9803) before deciding to allow advancement to the next dosing cohort in the dose escalation schedule. If no safety concerns were found, each dosing cohort was continued until the target enrollment of 37-38 patients was reached, and then the next dosing cohort was started. Each of the 4 dosing cohorts was designed to target enrollment of approximately 37-38 patients (25 randomized to active drug and 12-13 randomized to placebo). The DMC also reviewed safety data through hospital discharge after enrollment was completed in each dosing cohort. The primary analysis population was prespecified to be the modified intent-to-treat population based on randomized patients who received any quantity of study drug. All data analyses were performed independently by the trial coordinating center (Duke Clinical Research Institute, Durham, N.C.). Statistical comparisons were performed across dosing groups to explore whether a dose-response relationship exists among each dose of study drug compared with the respective concurrent placebo group. Furthermore, comparisons were also performed between pooled KAI-9803 dosing cohorts and pooled placebo cohorts to explore the potential activity of KAI-9803. For continuous end points, dose trends were assessed using the Jonckheere-Terpstra test. For dichotomous end points, dose trends were assessed using the Cochran-Mantel-Haenszel chi-square test of trend using ridit scores. Pair-wise contrasts were used to evaluate pooled KAI-9803 dosing cohorts versus pooled placebo using an ANOVA orthogonal test or the nonparametric Wilcoxon rank sum test. For categorical end points, Fisher's exact test or chisquare test was used to compare the KAI-9803 dose levels with pooled placebo.

Accordingly, this study demonstrates that, when administered via intracoronary injection during primary PCI for STEMI, KAI-9803, a δPKC inhibitor, has an acceptable safety and tolerability profile. Despite significant differences in patient clinical and presentation characteristics across dosing cohorts in this study, consistent improvements (non-statistically significant) in CK-MB AUC, ST-recovery AUC, and Technetium Tc-99m Sestamibi infarct size values with active study drug compared with concurrent placebo (except for infarct size values with the 5.0 mg dose) is consistent with potential drug activity.

### Utilization of Biomarkers of Reperfusion Success

Studies that have used multiple, simultaneous biomarkers of reperfusion success suggest that biomarker arrays provide a more comprehensive approach for the initial evaluation of adjunctive therapies for STEMI, both by enhancing information content and retrieval and by overcoming the loss of data that occurs when individual biomarkers are used. In the DELTA MI trial, multiple biomarkers of reperfusion success were used to evaluate the potential drug activity of KAI-9803, but consistent signs of drug activity across the array of biomarkers evaluated for this study were not demonstrated—a finding that may be accounted for by examining the design clinical trials that use biomarkers of reperfusion success. First, despite inclusion and exclusion criteria in DELTA MI that were designed to define a very specific, homogeneous patient population, factors that significantly influence biomarker end points—including total ischemia time before reperfusion, diabetes mellitus, prodromal angina before presentation, and collaterals to the LAD distribution—varied substantially among active study drug vs. concurrent placebo across dosing cohorts and influenced the evaluation of drug activity with the biomarker results. Second, a wide variability in the interquartile ranges of the quantitative biomarker results were observed due to the small sample sizes of the dosing cohorts, and this variability influenced the statistical comparisons between active study drug vs. concurrent placebo within dosing cohorts and prevented definitive conclusions regarding the impact of active study drug. Finally, consistent improvements (non-statistically significant) were demonstrated with active study drug were demonstrated with biomarkers collected during the first 24-36 hours such as ST recovery and CK-MB AUC values. However, biomarkers collected during later time points, such as infarct size measurements, were improved (non-statistically significant) with active study drug only in cohorts 1, 2, and 3, and no apparent differences were seen in left ventricular contractility assessments with echocardiography.

### Dose-Response Relationship

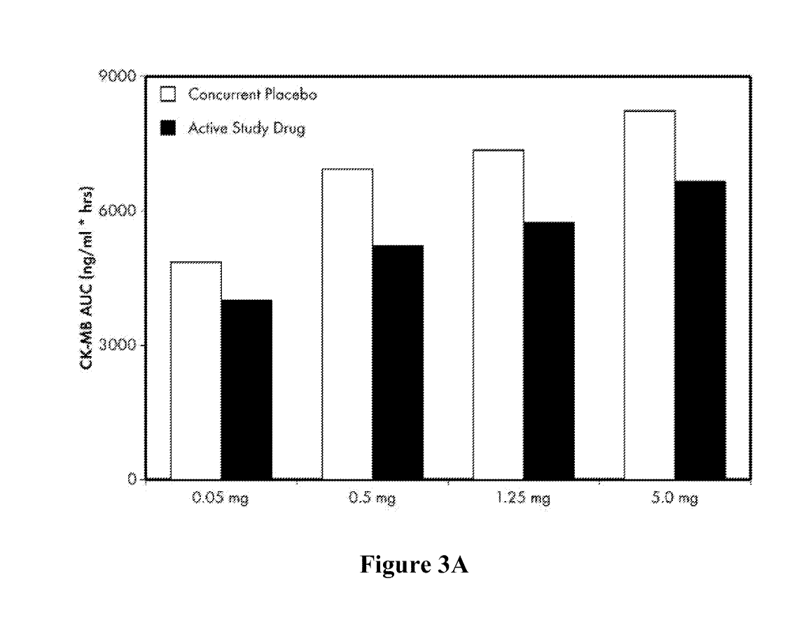

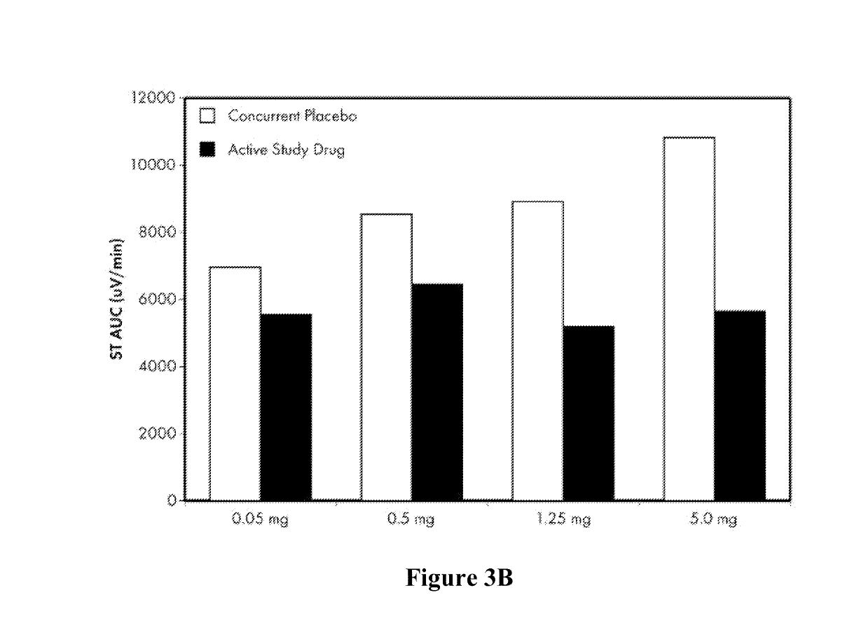

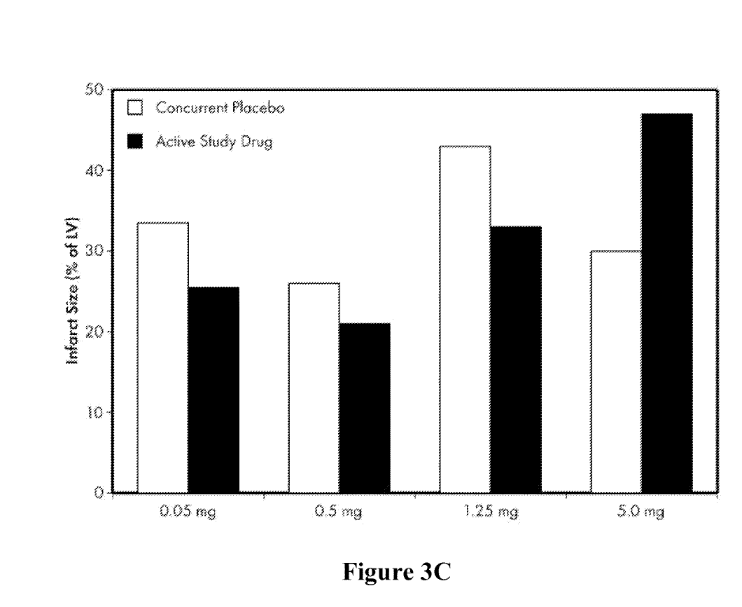

While KAI-9803 showed signs of potential drug activity in this early-phase trial, a dose-response relationship with escalating doses was not demonstrated. Possible explanations for this include (a) significant changes in patient recruitment, patient clinical characteristics, and presentation characteristics across the dosing cohorts limited comparisons among dose levels; (b) the initial dose tested (0.02 mg) during the first of the 2 injections in cohort 1 was equivalent to the dose used in the pre-clinical experiments that lead to a substantial reduction in infarct size, so higher doses may not have lead to incremental benefit but were tested to ensure safety and tolerability with dose escalation; and (c) the prolonged total ischemia times in the later dosing cohorts were associated with progressively worse biomarker results in the placebo-treated patients across dosing cohorts (Tables 4 and 5, FIG. 3). Because myocardial salvage and mortality reduction have been shown to be maximized when reperfusion occurs within the first 4 hours after symptom onset, there may be a “window of therapeutic opportunity” for an adjunctive therapy such as KAI-9803, beyond which irreversible myocardial damage may have occurred that cannot be ameliorated. As a result, the potential impact of larger doses of KAI-9803 tested in the later dosing cohorts may have been confounded by longer total ischemia times observed among patients treated with active study drug in cohorts 3 and 4 compared with the respective concurrent placebo patients in these cohorts (Table 2).

By targeting a precise molecular pathway involved in the myocardial and endothelial intracellular response to ischemia and reperfusion for patients with STEMI, 6-PKC inhibition with KAI-9803 represents a novel myocardial protection approach designed to optimize the results of reperfusion therapy with primary PCI.

1. A method for treating ST-elevation myocardial infarction, comprising:
identifying a subject suffering from ST-elevation myocardial infarction; and
administering to the subject via intracoronary delivery a therapeutically effective amount of a delta PKC isozyme-specific antagonist as an adjunct to primary percutaneous coronary intervention.

2. The method of claim 1, wherein the therapeutically effective amount is from about 0.1 to 5 mg.

3. The method of claim 2, wherein the therapeutically effective amount is about 0.5 mg.

4. The method of claim 2, wherein the therapeutically effective amount is about 1.25 mg.

5. The method of claim 2, wherein the therapeutically effective amount is about 4 mg.

6. The method according to claim 1, wherein the delta PKC isozyme-specific antagonist is KAI-9803.
